(19) United States
(12) Patent Application Publication
(10) Pub. No.: US 2026/0226097 A1
(43) Pub. Date: Aug. 6, 2026

(54) EXTRACTION OF NUCLEAR AND CHROMATIN-BOUND PROTEINS USING VOLATILE SALTS

(71) Applicant: TALUS BIOSCIENCE, INC., Seattle, WA (US)

(72) Inventors: Daniele CANZANI, Seattle, WA (US); Andrea I. GUTIERREZ, Seattle, WA (US); Lindsay K. PINO, Seattle, WA (US); Alexander J. FEDERATION, Renton, WA (US)

(21) Appl. No.: 19/152,833

(22) PCT Filed: Feb. 2, 2024
(86) PCT No.: PCT/US2024/014337

§ 371 Date: Jul. 31, 2025

Related U.S. Application Data
(60) Provisional application No. 63/443,216, filed on Feb. 3, 2023.

(51) Int. Cl. C07K 1/14; G01N 33/68

(52) U.S. Cl. CPC C07K 1/145; G01N 33/6848

(57) ABSTRACT

The present disclosure relates to methods and compositions for peptide and protein extraction using solutions and buffers that contain volatile salts which are compatible with analysis by mass spectrometry.

### CROSS-REFERENCES

This application claims the benefit of and priority to U.S. Provisional Patent Application No. 63/443,216, filed on Feb. 3, 2023, the entire contents of which are incorporated by reference herein.

### FIELD OF THE INVENTION

The present disclosure relates generally to methods and compositions for using buffers and solutions that contain volatile salts to extract and fractionate proteins from cells.

### BACKGROUND OF THE INVENTION

Detecting the presence and abundance of peptides and proteins in different cell compartments during healthy and diseased states is a critical tool in understanding biological processes and how their malfunction contributes to illness and diseases. Mass spectrometry (MS) is commonly used to analyze protein samples to identify and quantify the peptides and proteins that are present in the samples. To extract proteins from cell systems for MS analysis, buffers that contain non-volatile salts (e.g., NaCl) are frequently used. However, these non-volatile salts must be removed prior to analysis of protein or peptide samples for mass spectrometry (MS) analysis due to their interference with electrospray ionization (ESI). Sample clean-up steps to remove these salts are time consuming, costly, and may reduce peptide and protein yields during sample processing. Therefore, there is a need in the field to improve methods of peptide and protein extraction for MS analysis. The present disclosure addresses this need by providing improved methods and compositions for peptide and protein extraction.

### SUMMARY OF THE INVENTION

The present disclosure provides methods of extracting proteins from nuclei and chromatin, comprising contacting nuclei and/or chromatin with a solution comprising a volatile salt, separating insoluble chromatin from the solution, and collecting the solution supernatant, wherein the solution supernatant comprises proteins from nuclei and/or chromatin.

In some embodiments of the methods of extracting proteins from nuclei and chromatin, the volatile salt is ammonium acetate.

In some embodiments of the methods of extracting proteins from nuclei and chromatin, the extracted proteins are analyzed by mass spectrometry.

In some embodiments of the methods of extracting proteins from nuclei and chromatin, the volatile salt is not removed from the extracted proteins before their analysis by mass spectrometry.

In some embodiments of the methods of extracting proteins from nuclei and chromatin, the extracted proteins comprise fragments, e.g., peptide fragments, of full-length proteins.

In some embodiments of the methods of extracting proteins from nuclei and chromatin, the nuclei are separated from the cells before the nuclei and/or chromatin are contacted with the volatile salt.

In some embodiments of the methods of extracting proteins from nuclei and chromatin, samples enriched in nucleoplasm associated proteins, euchromatin associated proteins, and/or heterochromatin associated proteins are isolated from cells, the method comprising:
(a) lysing the cells;
(b) separating nuclei from the cells following (a); and
(c) extracting nucleoplasm associated proteins, euchromatin associated proteins, and/or heterochromatin associated proteins from the separated nuclei by contacting nuclei and/or chromatin with a solution comprising a volatile salt, separating insoluble chromatin, nuclear membrane, and/or non-chromatin proteins from the solution, and collecting the solution supernatant, wherein the solution supernatant comprises nucleoplasm associated proteins, euchromatin associated proteins, and/or heterochromatin associated proteins.

In some embodiments, the solution comprises 500-1000 mM or 550-1000 mM of the volatile salt, e.g., ammonium acetate. In some embodiments, the solution comprises about 500 mM, about 550 mM, about 600 mM, about 650 mM, or about 700 mM of the volatile salt, e.g., ammonium acetate.

In some embodiments of the methods of extracting proteins from nuclei and chromatin, samples enriched in nucleoplasm associated proteins, euchromatin associated proteins, and/or heterochromatin associated proteins are isolated from cells, the method comprising:
(a) contacting cells with a detergent solution to lyse the cells;
(b) separating nuclei from the cells (e.g., the lysed cells) following (a); and
(c) extracting the nucleoplasm associated proteins, euchromatin associated proteins, and/or heterochromatin associated proteins from the separated nuclei as follows:
   (i) re-suspending the nuclei of (b) in a first solution comprising a first concentration of a volatile salt to extract nucleoplasm associated proteins, euchromatin associated proteins, and/or heterochromatin associated proteins;
   (ii) separating insoluble chromatin, nuclear membrane, and/or non-chromatin proteins after (i); and
   (iii) collecting a first supernatant liquid after (ii), wherein the first supernatant liquid is enriched for nucleoplasm associated proteins, euchromatin associated proteins, and/or heterochromatin associated proteins,
   thereby isolating a first sample enriched in enriched for nucleoplasm associated proteins, euchromatin associated proteins, and/or heterochromatin associated proteins.

In some embodiments, the method further comprises obtaining cytoplasmic proteins following step (b). In some embodiments, the solution comprises 500-1000 mM or 550-1000 mM of the volatile salt, e.g., ammonium acetate. In some embodiments, the solution comprises about 500 mM, about 550 mM, about 600 mM, about 650 mM, or about 700 mM of the volatile salt, e.g., ammonium acetate.

In some embodiments of the methods of extracting proteins from nuclei and chromatin, samples enriched in nucleoplasm associated proteins, euchromatin associated proteins, and/or heterochromatin associated proteins are isolated from cells, the method comprising:
(a) contacting cells with a detergent solution to lyse the cells;
(b) separating nuclei from the cells (e.g., the lysed cells) following (a); and
(c) sequentially extracting the nucleoplasm associated proteins, euchromatin associated proteins, and/or heterochromatin associated proteins from the separated nuclei as follows:
   (i) re-suspending the nuclei of (b) in a first solution comprising a first concentration of a volatile salt to extract nucleoplasm associated proteins;
   (ii) separating insoluble chromatin, nuclear membrane, and/or non-chromatin proteins after (i);
   (iii) collecting a first supernatant liquid after (ii), wherein the first supernatant liquid is enriched for nucleoplasm associated proteins;
   (iv) re-suspending the insoluble chromatin of (ii) in a second solution comprising a second concentration of the volatile salt to extract euchromatin associated proteins;
   (v) separating insoluble chromatin after (iv);
   (vi) collecting a second supernatant liquid after (v), wherein the second supernatant liquid is enriched for euchromatin associated proteins;
   (vii) re-suspending the insoluble chromatin of (v) in a third solution comprising a third concentration of the volatile salt to extract heterochromatin associated proteins;
   (viii) separating insoluble chromatin after (vii); and
   (ix) collecting a third supernatant liquid after (viii), wherein the third supernatant liquid is enriched for heterochromatin associated proteins,
   thereby isolating a first sample enriched in nucleoplasm associated proteins, a second sample enriched in euchromatin associated proteins, and a third sample enriched in heterochromatin associated proteins from the cells.

In some embodiments, the method further comprises obtaining cytoplasmic proteins following step (b).

In some embodiments of the methods of extracting proteins from nuclei and chromatin, the cells, nuclei, and chromatin are not contacted with a non-volatile salt.

In some embodiments of the methods of extracting proteins from nuclei and chromatin, the volatile salt is not removed from the supernatant liquid.

In some embodiments of the methods of extracting proteins from nuclei and chromatin, the volatile salt is ammonium acetate, ammonium bicarbonate, trimethylammonium acetate, or trimethylammonium bicarbonate.

In some embodiments of the methods of extracting proteins from nuclei and chromatin, the volatile salt is ammonium acetate.

In some embodiments of the methods of extracting proteins from nuclei and chromatin, the solutions used to extract the proteins comprise:
(a) a first solution comprising 50-150 mM of a volatile salt;
(b) a second solution comprising 200-500 mM of a volatile salt; and
(c) a third solution comprising 500-1000 mM or 550-1000 mM of a volatile salt, e.g., ammonium acetate.

In some embodiments of the methods of extracting proteins from nuclei and chromatin, the solution(s) comprises about 500-1000 mM of the volatile salt, e.g., ammonium acetate.

In some embodiments of the methods of extracting proteins from nuclei and chromatin, the solution(s) comprises about 500 mM, about 550 mM, about 600 mM, about 650 mM, or about 700 mM of the volatile salt, e.g., ammonium acetate.

In some related embodiments of the methods of extracting proteins from nuclei and chromatin, the solution(s) further comprises 0.1-1% of a detergent.

In some related embodiments of the methods of extracting proteins from nuclei and chromatin, the detergent in the solution(s) is selected from Triton X-100, NP-40, and SDS.

The present disclosure further provides methods for comparing the proteins present in the nucleus and/or chromatin-associated proteins of two or more different cell populations, comprising:
(a) extracting proteins from the nucleus and/or chromatin of a first cell population according to any of the disclosed methods of extracting proteins from nuclei and chromatin using a solution that comprise a volatile salt;
(b) extracting proteins from the nucleus and/or chromatin of a second cell population according to any of the disclosed methods of extracting proteins from nuclei and chromatin using a solution that comprise a volatile salt; and
(c) analyzing the proteins obtained from the first cell population and the second cell population,
thereby comparing the proteins present in the nucleus and/or chromatin-associated proteins of the two or more different cell populations. In some embodiments, the method further comprises obtaining cytoplasmic proteins from each cell population.

In some embodiments of the methods for comparing the proteins present in the nucleus and/or chromatin-associated proteins of two or more different cell populations, the first cell population and the second cell population were exposed to different conditions. In some related embodiments, the different conditions comprise one or more of: different growth or culture conditions; different environmental conditions; and presence or absence of, or different, genomic or cellular modifications.

The present disclosure also provides methods of extracting proteins from nuclei, comprising contacting nuclei bound to a solid support with a solution comprising a volatile salt, e.g., ammonium acetate, and then separating the nuclei bound to the solid support from the solution, wherein following the contacting, the solution comprises nuclear and/or chromatin-associated proteins.

In some related embodiments, the volatile salt is ammonium acetate.

In some embodiments of the methods of extracting proteins from nuclei, comprising contacting nuclei bound to a solid support with a solution comprising a volatile salt, the extracted proteins are analyzed by mass spectrometry.

In some embodiments of the methods of extracting proteins from nuclei, comprising contacting nuclei bound to a solid support with a solution comprising a volatile salt, the volatile salt is not removed from the extracted proteins before the analysis by the mass spectrometry

In some embodiments of the methods of extracting proteins from nuclei, comprising contacting nuclei bound to a solid support with a solution comprising a volatile salt, the extracted proteins comprise fragments, e.g., peptide fragments, of full-length proteins

In some embodiments of the methods of extracting proteins from nuclei, comprising contacting nuclei bound to a solid support with a solution comprising a volatile salt, the nuclei are separated from cells before contacting the volatile salt.

In some embodiments of the methods of extracting proteins from nuclei, comprising contacting nuclei bound to a solid support with a solution comprising a volatile salt, samples enriched in nucleoplasm associated proteins, euchromatin associated proteins, and/or heterochromatin associated proteins are prepared from cells, the method comprising:
(a) contacting cells with a detergent solution to lyse the cells;
(b) binding the nuclei to a solid support and separating nuclei from the cells following (a); and
(c) extracting nucleoplasm associated proteins, euchromatin associated proteins, and/or heterochromatin associated proteins from the separated nuclei as follows:
   (i) contacting the nuclei bound to the solid support following (b) with a solution comprising a volatile salt to extract nucleoplasm associated proteins, euchromatin associated proteins, and/or heterochromatin associated proteins; and
   (ii) separating the nuclei bound to the solid support from the first solution, wherein the first solution comprises nucleoplasm associated proteins, euchromatin associated proteins, and/or heterochromatin associated proteins following (i); thereby isolating a sample enriched in nucleoplasm associated proteins, euchromatin associated proteins, and/or heterochromatin associated proteins from the cells.

In particular embodiments, the solution comprises about 500-1000 mM of the volatile salt, e.g., ammonium acetate. In some embodiments the solution comprises about 500 mM, about 550 mM, about 600 mM, about 650 mM, or about 700 mM of the volatile salt, e.g., ammonium acetate. In some embodiments, the method further comprises obtaining cytoplasmic proteins.

In some embodiments of the methods of extracting proteins from nuclei, comprising contacting nuclei bound to a solid support with a solution comprising a volatile salt, samples enriched in nucleoplasm associated proteins, euchromatin associated proteins, and/or heterochromatin associated proteins are prepared from cells, the method comprising:
(a) contacting cells with a detergent solution to lyse the cells;
(b) binding the nuclei to a solid support and separating nuclei from the cells following (a); and
(c) sequentially extracting the nucleoplasm associated proteins, euchromatin associated proteins, and/or heterochromatin associated proteins from the separated nuclei as follows:
   (i) contacting the nuclei bound to the solid support following (b) with a first solution comprising a first concentration of a volatile salt to extract nucleoplasm associated proteins;
   (ii) separating the nuclei bound to the solid support from the first solution, wherein the first solution comprises nucleoplasm associated proteins following (i);
   (iii) contacting the nucleic bound to the solid support with a second solution comprising a second concentration of the volatile salt to extract euchromatin associated proteins;
   (iv) separating the nuclei bound to the solid support from the second solution, wherein the second solution comprises euchromatin associated proteins following (iii);
   (v) contacting the nucleic bound to the solid support with a third solution comprising a third concentration of the volatile salt to extract heterochromatin associated proteins; and
   (vi) separating the nuclei bound to the solid support from the third solution, wherein the second solution comprises heterochromatin associated proteins following (v); thereby isolating a first sample enriched in nucleoplasm associated proteins, a second sample enriched in euchromatin associated proteins, and a third sample enriched in heterochromatin associated proteins from the cells.

In some embodiments, the method further comprises obtaining cytoplasmic proteins.

In some embodiments of the methods of extracting proteins from nuclei, comprising contacting nuclei bound to a solid support with a solution comprising a volatile salt, the nuclei are not contacted with a non-volatile salt.

In some embodiments of the methods of extracting proteins from nuclei, comprising contacting nuclei bound to a solid support with a solution comprising a volatile salt, the volatile salt is not removed from the samples.

In some embodiments of the methods of extracting proteins from nuclei, comprising contacting nuclei bound to a solid support with a solution comprising a volatile salt, the volatile salt is ammonium acetate, ammonium bicarbonate, trimethylammonium acetate, or trimethylammonium bicarbonate.

In some embodiments of the methods of extracting proteins from nuclei, comprising contacting nuclei bound to a solid support with a solution comprising a volatile salt, the volatile salt is ammonium acetate

In some embodiments of the methods of extracting proteins from nuclei, comprising contacting nuclei bound to a solid support with a solution comprising a volatile salt, the solution may be:
(a) a first solution comprising 50-150 mM of the volatile salt;
(b) a second solution comprising 200-500 mM of the volatile salt; or
(c) a third solution comprising 500-1000 mM or 550-1000 mM of the volatile salt.

The present disclosure further provides methods for comparing the proteins present in the nucleus and/or chromatin-associated proteins of two or more different cell populations, comprising:
(a) extracting proteins from the nucleus and/or chromatin of a first cell population according to any of the disclosed methods of extracting proteins from nuclei bound to a solid support with a solution comprising a volatile salt;
(b) extracting proteins from the nucleus and/or chromatin of a second cell population according to any of the disclosed methods of extracting proteins from nuclei bound to a solid support with a solution comprising a volatile salt; and
(c) analyzing the proteins obtained from the first cell population and the second cell population, thereby comparing the proteins present in the nucleus and/or chromatin-associated proteins of the two or more different cell populations. In some embodiments, the method further comprises obtaining cytoplasmic proteins for each cell population.

In some related embodiments, the first cell population and the second cell population were exposed to different conditions

In some related embodiments, the different conditions comprise one or more of: different growth or culture conditions; different environmental conditions; and presence or absence of, or different, genomic or cellular modifications

In some embodiments of the methods of the present disclosure, the solid support comprises one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc) present on glycoproteins in the nuclear membrane of the nuclei.

In some embodiments, the one or more lectin comprises Erythrina crista-galli lectin (ECL), Ricinus communis agglutinin (RCA), e.g., RCAI or RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin.

In some embodiments of the methods of the present disclosure, the solid support is a bead.

In some embodiments of the methods of the present disclosure, the solid support is a magnetic bead.

In some embodiments of the methods of the present disclosure, the nuclei are contacted with the solid support while in a sample comprising cells, cell lysate, and/or other non-nuclear cellular components.

In some embodiments of the methods of the present disclosure, wherein the solid support is magnetic, the solid support and associated nuclei are transferred or removed, e.g., separated from cells, cell lysate, and/or other non-nuclear cellular components, using a magnet.

In some embodiments of the methods of the present disclosure, the method is a high-throughout method, wherein the nuclei are present in a first solution within multiple wells of a sample plate, the solid support is a magnetic bead, and nuclei bound to the solid support are transferred or removed from the multiple wells using magnets.

In some related embodiments, following transfer or removal from the multiple wells comprising the first solution, the nuclei are placed into a second solution within multiple wells of a sample plate, and the nuclei bound to the solid support are then transferred or removed from the multiple wells using magnets.

In some related embodiments, following transfer or removal from the multiple wells comprising the second solution, the nuclei are placed into a third solution within multiple wells of a sample plate, and the nuclei bound to the solid support are then transferred or removed from the multiple wells using magnets.

In some related embodiments, the first solution is an isotonic solution to extract nucleoplasm proteins, the second solution is a low salt solution to extract euchromatic proteins, and the third solution is a high salt solution to extract heterochromatin proteins.

### BRIEF DESCRIPTION OF THE DRAWINGS

FIG. 1 provides a schematic of how buffers containing volatile salts can be used to extract the following protein fractions: cytosolic fraction, nucleoplasm fraction, euchromatin fraction, and heterochromatin fraction.

FIGS. 2A-2B show the average peptide (FIG. 2A) and protein (FIG. 2B) counts detected from liquid chromatography-mass spectrometry (LC-MS) experiments analyzing the following protein fractions in triplicates: cytoplasm, euchromatin, heterochromatin, and nucleoplasm. The protein fractions were extracted using buffers containing ammonium acetate, a volatile salt.

FIG. 3 shows the GO (gene ontology) enrichment analysis of four protein fractions (cytoplasm, euchromatin, heterochromatin, and nucleoplasm) isolated using buffers containing ammonium acetate, a volatile salt, and analyzed by LC-MS. For each cellular component listed below the x-axis, the four bars from left to right correlate to cytoplasm protein fraction, euchromatin protein fraction, heterochromatin protein fraction, and nucleoplasm protein fraction, respectively.

FIGS. 4A-4B provide Venn diagrams that show the overlap of peptides (FIG. 4A) and proteins (FIG. 4B) that were detected by LC-MS using either ammonium acetate containing buffers (AA), or conventional buffers that contain NaCl (NaCl).

FIG. 5 shows the correlation of protein abundances in samples extracted using ammonium acetate (AA)-based extraction buffers and protein abundances in samples extracted using NaCl-based extraction buffers, quantified by LC-MS, of each of the following protein fractions: cytoplasm (CYTO), euchromatin (EUCH), heterochromatin (HET), and nucleoplasm (NUC).

FIG. 6 provides glycomic mass spectrometry results in a volcano plot showing the enriched N-glycans in the cytosolic (containing the outer cell membrane) and LacNAc-containing glycans in the insoluble (containing the nuclear membrane) fractions of MM1.S cells that were subjected to chromatin extraction.

FIG. 7 provides a schematic of how lectin-conjugated magnetic beads can be used to extract proteins from cell nuclei. Nuclei are washed with buffers that contain increasing concentrations of NaCl, which separates the protein content into nuclear, euchromatin (EUCH), and heterochromatin (HET) fractions.

FIGS. 8A-8D show representative images of nucleoplasm fractions taken using an EVOS FLoid imaging system. From left to right, the panels show images taken using blue light (BL), images taken using white light (brightfield; WL), and an overlaid image of the blue and white light images. FIG. 8A and FIG. 8B show images of the nucleoplasm fraction prepared using ConA-conjugated magnetic beads from two different samples. FIG. 8C shows images of the nucleoplasm fraction from a sample prepared using RCA120-conjugated magnetic beads. FIG. 8D shows images of the nucleoplasm fraction from a sample prepared using ECL-conjugated magnetic beads. FIG. 8E provides the results of protein quantification by 660 assay of the euchromatin fraction and heterochromatin fraction isolated from 4 million MMS. 1 cells using ConA, ECL, or RCA-conjugated magnetic beads. For each of the euchromatin fraction and heterochromatin fraction, the bars from left to right are results using magnetic beads conjugated to ConA, RCA, and ECL, respectively. Significant differences are indicated by “**”

FIG. 9 shows the GO (gene ontology) enrichment analysis of proteins isolated using ConA, ECL, or RCA-conjugated magnetic beads, which demonstrates that RCA and ECL are superior for extracting nuclear proteins from cell nuclei. For each cell component, the bars from left to right are results using magnetic beads conjugated to ConA, RCA, and ECL, respectively.

FIG. 10 provides a schematic for high-throughput nuclear protein fractionation using a liquid handling robot with a magnetic head. The robot automates the use of lectin-conjugated magnetic beads to isolate separate nuclear fractions such as nucleoplasm, euchromatin, and heterochromatin fractions.

FIGS. 11A-11C show the peptides (FIG. 11A) and proteins (FIG. 11B) that were detected by LC-MS from three biological replicates of chromatin extracted from THP-1 nuclei using ECL-conjugated magnetic beads. FIG. 11C shows the coefficient of variation (CV) of protein abundances across the three biological replicates in this experiment.

FIGS. 12A-12F show protein abundances of bromodomain-containing protein 4 (BRD4) (FIG. 12A), bromodomain-containing protein 3 (BRD3) (FIG. 12B), bromodomain-containing protein 2 (BRD2) (FIG. 12C), menin (MEN1) (FIG. 12D), histone H4 (H4) (FIG. 12E), and beta-actin (ACTB) (FIG. 12F) in three drug treatment conditions from three replicates of LC-MS experiments analyzing protein fractions that were extracted from THP-1 cell nuclei by automation with the KingFisher™ robot using ECL-conjugated magnetic beads.

### DETAILED DESCRIPTION OF THE INVENTION

To extract proteins from cell systems for mass spectrometry (MS) analysis, buffers that contain non-volatile salts (e.g., NaCl) are frequently used. Common desalting methods (such as those found in Varnavides et. al. In Search of a Universal Method: A Comparative Survey of Bottom-Up Proteomics Sample Preparation Methods. J Proteome Res. 2022 Oct. 7; 21 (10): 2397-2411) for removal of these non-volatile salts in preparation for MS analysis are time consuming, costly, and may reduce peptide and protein yields during sample processing.

The present disclosure provides methods of extracting protein fractions from cell samples using solutions or buffers that contain volatile salts compatible with MS-based analysis, negating the need for the desalting step. Generally, the methods described herein include contacting cells or components or subfractions thereof (referred to as sample or cell sample) with solutions or buffers that comprise varying concentrations of volatile salts. In some embodiments, the samples are not contacted with solutions or buffers containing non-volatile salts. In some embodiments, the sample comprises live cells. In some embodiments, the sample comprises cell nuclei. In some embodiments, the volatile salt buffers of the present disclosure are used in methods of nuclear protein extractions using lectin-coupled solid supports.

Isolation of cell nuclei is useful in the fields of genetics, genomics, proteomics, and metabolomics and in a variety of biomedical applications. However, handling cell nuclei, including moving, immobilizing, isolating, enriching, or extracting, is difficult. Current methods to isolate or enrich cell nuclei are sub-optimal, often leading to contamination with other unwanted cell structures. As such, there is considerable room for improvement in methods that are effective and efficient for separation of cell nuclei from other cell structures. The methods disclosed herein related to the use of volatile salts to extract proteins may be combined with methods disclosed herein for handling and isolating cell nuclei and components thereof through targeting the sugar groups found on glycoproteins present in the nuclear membrane.

### Methods

### Solutions and Buffers

As used herein, the term “solution” or “solutions” refers to a liquid mixture, wherein one or more solute is distributed in one or more solvent. As used herein, the term “buffer” or “buffers” refer to a solution that is meant to maintain a certain pH or pH range. Generally, the buffer comprises a weak acid or a weak base. The solutions and buffers in the present disclosure comprise one or more volatile salt, wherein the volatile salt is compatible with MS-based analysis. In some embodiments, the one or more volatile salt does not interfere with electrospray ionization (ESI). In some embodiments, the one or more volatile salt comprises ammonium acetate, ammonium bicarbonate, trimethylammonium acetate, or trimethylammonium bicarbonate. In some embodiments, the one or more volatile salt is selected from ammonium acetate, ammonium bicarbonate, trimethylammonium acetate, or trimethylammonium bicarbonate. In some embodiments, the one or more volatile salt comprises ammonium acetate. In some embodiments, the volatile salt is ammonium acetate. In some embodiments, the volatile salt is not removed prior to sample analysis by mass spectrometry.

Methods disclosed herein contemplate the use of different buffers suitable for extracting proteins enriched in or associated with different nuclear and/or chromatin fractions, such as nucleoplasm, euchromatin, and heterochromatin.

In certain embodiments, an isotonic buffer is used to extract nucleoplasm-associated proteins. In particular embodiments, the isotonic buffer comprises a volatile salt at a concentration of about 50 mM to 150 mM. In some embodiments, an isotonic buffer comprising one or more of the following volatile salts: ammonium acetate, ammonium bicarbonate, trimethylammonium acetate, or trimethylammonium bicarbonate. In some embodiments, the isotonic buffer comprises ammonium acetate. In some embodiments, an isotonic buffer has approximately physiological salt concentrations. In some embodiments, an isotonic buffer comprises a volatile salt concentration of about 10 mM to about 150 mM. In some embodiments, an isotonic buffer comprises an ammonium acetate concentration of about 10 mM to about 150 mM. In some embodiments, an isotonic buffer comprises a volatile salt concentration, e.g., an ammonium acetate concentration, of about 5 mM, about 10 mM, about 15 mM, about 20 mM, about 25 mM, about 50 mM, about 75 mM, about 100 mM, about 125 mM, or about 150 mM. In some embodiments, an isotonic buffer comprises an ammonium acetate concentration of about 15 mM. In some embodiment, the isotonic buffer comprises 10 mM Tris pH 8.0, 60 mM KCl, 1.5 mM EDTA, and 15 mM ammonium acetate.

In certain embodiments, a low salt buffer is used to extract euchromatin-associated proteins, alone or together with nucleoplasm-associated proteins. In particular embodiments, the low salt buffer comprises a volatile salt at a concentration of about 200 mM to about 500 mM. In certain embodiments, a low salt buffer comprises one or more of the following volatile salts: ammonium acetate, ammonium bicarbonate, trimethylammonium acetate, or trimethylammonium bicarbonate. In some embodiments, the low salt buffer comprises ammonium acetate. In some embodiments, the low salt buffer comprises a volatile salt concentration of about 200 mM to about 500 mM. In some embodiments, the low salt buffer comprises a volatile salt concentration, e.g., an ammonium acetate concentration, of about 200 mM, about 225 mM, about 250 mM, about 275 mM, about 300 mM, about 350 mM, about 400 mM, about 450 mM, or about 500 mM. In some embodiments, the low salt buffer comprises an ammonium acetate concentration of about 250 mM. In some embodiments, the low salt buffer comprises 10 mM Tris pH 8.0, 1.5 mM EDTA, and 250 mM ammonium acetate.

In certain embodiments, a high salt buffer is used to extract heterochromatin-associated proteins, alone or together with euchromatin-associated proteins or nucleoplasm-associated proteins and euchromatin-associated proteins. In particular embodiments, the high salt buffer comprises a volatile salt at a concentration of about 500 mM to about 1000 mM or about 550 mM to about 1000 mM. In some embodiments, a high salt buffer comprises one or more of the following volatile salts: ammonium acetate, ammonium bicarbonate, trimethylammonium acetate, or trimethylammonium bicarbonate. In some embodiments, the high salt buffer comprises ammonium acetate. In some embodiments, the high salt buffer comprises a volatile salt concentration of greater than about 500 mM, e.g., about 500 mM to about 1,000 mM or about 550 mM to about 1000 mM. In some embodiments, the high salt buffer comprises a volatile salt concentration, e.g., an ammonium acetate concentration, of about 550 mM, about 575 mM, about 600 mM, about 650 mM, about 700 mM, about 750 mM, about 800 mM, about 900 mM, or about 1000 mM. In some embodiments, the high salt buffer comprises an ammonium acetate concentration of about 600 mM. In some embodiments, the high salt buffer comprises 10 mM Tris pH 8.0, 1.0 mM EDTA, and 600 mM ammonium acetate.

### Cells

The cells may be from a variety of sources. For examples, the cells may be cell lines, including primary cell lines, or they may be obtained from a tissue, organ, or organism, e.g., mammalian cells. Cells may also be present in cultured organoids, e.g., in vitro organoids produced from cells obtained from a cell, tissue, organ, or organism, e.g., a mammal.

In some embodiments, the cells are mammalian cells or are obtained from a mammal. The mammalian cells may be any cell type. In some embodiments, the mammalian cell is an epithelial cell, a connective tissue cell, a hormone secreting cell, a nerve cell, a skeletal muscle cell, a blood cell, an immune system cell, or a stem cell.

In some embodiments, the cells are obtained from blood, serum, urine, stool, saliva, lymph fluid, cerebrospinal fluid, synovial fluid, cystic fluid, ascites, pleural effusion, amniotic fluid, chorionic villus sample, vaginal fluid, interstitial fluid, nasal swab sample, buccal swab sample, sputum, bronchial lavage, Pap smear sample, or ocular fluid. The cell sample may comprise cells obtained from a blood sample, an aspirate sample, or a smear sample. Cells may be obtained from a biopsy sample.

In some embodiments, the cells are derived from a cell line. illustrative cell lines include, but are not limited to, 293A cell line, 293 FT cell line, 293F cell line, 293 H cell line, HEK 293 cell line, CHO DG44 cell line, CHO-S cell line, CHO-K1 cell line, Expi293F.TM. cell line, Flp-In. TM. T-REX. TM. 293 cell line, Flp-In. TM.-293 cell line, Flp-In. TM.-3T3 cell line, Flp-In. TM.-BHK cell line, Flp-In. TM.-CHO cell line, Flp-In. TM.-CV-1 cell line, Flp-In. TM.-Jurkat cell line, FreeStyle. TM. 293-F cell line, FreeStyle. TM. CHO-S cell line, GripTite. TM. 293 MSR cell line, GS-CHO cell line, HepaRG.TM. cell line, T-REx. TM. Jurkat cell line, Per.C6 cell line, T-REX. TM.-293 cell line, T-REx. TM.-CHO cell line, T-REX. TM.-HeLa cell line, NC-HIMT cell line, and PC12 cell line.

The cells may comprise healthy and/or diseased or damaged cells. For example. In some embodiments, the cells are obtained from a healthy mammal or from a mammal diagnosed with a disease or disorder, such as, e.g., a cancer or tumor, an inflammatory disease or disorder, an immune disease or disorder, a genetic disease or disorder, a metabolic disease or disorder, a cardiac disease or disorder, ischemia or reperfusion injury, or an infection, e.g., infection by bacteria, virus, fungi, etc. Cells may be obtained from a healthy or a diseased tissue or organ.

A cell sample may comprise cancerous cells. The cancerous cells may form a cancer which may be a solid tumor or a hematologic malignancy. The cancerous cell sample may comprise cells obtained from a solid tumor. The solid tumor may include a sarcoma or a carcinoma. Exemplary sarcoma cell sample may include, but are not limited to, cell sample obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (WE), malignant fibrous histiocytoma (WE) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Illustrative carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some embodiments, the cancerous cell sample may comprise cells obtained from a hematologic malignancy. Hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Exemplary B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the cell sample may comprise circulating tumor cells. In some embodiments, a circulating tumor cell sample may comprise lymphoma cells, fetal cells, apoptotic cells, epithelia cells, endothelial cells, stem cells, progenitor cells, mesenchymal cells, osteoblast cells, osteocytes, hematopoietic stem cells, foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a lung, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

In some embodiments, a cell sample may be a peripheral blood mononuclear cell sample or comprise peripheral blood mononuclear cells.

Cell samples (such as a biopsy sample) may be obtained from a mammal by any suitable means of obtaining the sample using well-known and routine clinical methods. For example, procedures for drawing and processing tissue samples such as from a needle aspiration biopsy are well-known and may be employed to obtain a sample for use in the methods provided. Typically, for collection of such a tissue sample, a thin hollow needle is inserted into a mass such as a tumor mass for sampling of cells that, after being stained, will be examined under a microscope.

In some embodiments, a cell sample may comprise cells of a tumor cell line. Illustrative tumor cell lines include, but are not limited to, cell samples from tumor cell lines such as MM1.S, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

### Nuclei

As used herein, the terms “nuclei” and “cell nuclei” are used interchangeably and refer to intact cell nuclei and/or any of one or more component thereof which make up the nuclei. Such components include, but are not limited to, the nuclear membrane, the nuclear envelope, the nuclear lamina, chromatin, and the nucleolus.

### Cell Processing and Protein Extraction

Protein extraction may be accomplished through chromatin enriching salt separation using volatile salt-based buffers and solutions.

Cells may be washed, harvested, and/or lysed, e.g., to extract proteins from other cellular material. In some embodiments, nuclei are isolated from cells, and proteins extracted from the isolated nuclei. In preparation for cell nuclei isolation, cells may be harvested by centrifugation, and washed, e.g., with PBS, and then re-centrifuged. In some embodiments, cells are centrifuged at 400 RCF for 5 minutes at 4° C. In some embodiments, the cells are washed with ice cold 1×PBS. In some embodiments, the cells are washed more than one time with PBS.

Cells (e.g., pelleted cells) may be lysed by methods known in the art, including but not limited to, detergent-based lysis, freeze-thaw lysis, mechanical disruption of membranes, enzymatic digestion, sonication, and osmotic shock. In some embodiments, the pelleted cells are resuspended in a first suspension buffer, wherein the resuspended cells are lysed by this first suspension buffer. In some embodiments, this first suspension buffer comprises a detergent. The detergent may be ionic or non-ionic. In some embodiments, the detergent comprises NP40. In some embodiments, the detergent comprises SDS, TritonX100, and/or Tween20. The detergent may be a mass-spectrometry acid-labile detergent. In some embodiments, the concentration of the detergent in the first suspension buffer is up to 4%. In some embodiments, the concentration of the detergent ranges from 0.01% to 4% or from 0.1% to 4%. For example, the detergent may be at a concentration of 0.01% to 0.1%. In some embodiments, the first suspension buffer is a nuclear extraction buffer (NEB). In some embodiment the first suspension buffer is Nuclear Extraction Buffer (NEB) comprising 0.1% Triton X-100. The cells are contacted with the first suspension buffer for a time sufficient to lyse the cells. In some embodiments, the period of contact is at least 10 minutes. In some embodiments, other methods may be used to lyse the cells, such as, e.g., sonication or physical cutting or mincing. In some embodiments, cell lysis is performed on a cold block, on wet ice, or at 4° C.

In some embodiments, after cell lysis, the samples are centrifuged. In some embodiments, the samples are centrifuged at 400 RCF for 5 minutes at 4° C. to generate a pellet comprising nuclei and chromatin, e.g., insoluble chromatin, nuclear membrane, and/or non-chromatin proteins. The supernatant is then removed. In some embodiments, this supernatant is discarded. In some embodiments, this supernatant can be retained as the Cytosolic Fraction.

In some embodiments, after removal of the supernatant containing cytosolic proteins, the pellets are re-suspended in a suspension solution comprising a volatile salt. In some embodiments, the concentration of the volatile salt is used to selectively extract one or more subset of nuclear proteins, e.g., one or more of: nucleoplasm-enriched proteins, euchromatin-enriched proteins, and/or heterochromatin-enriched proteins. In one embodiment, the nuclei may be re-suspended and incubated in an isotonic buffer to extract nucleoplasm-enriched proteins, and then centrifuged to facilitate collection supernatant as a Nucleoplasm Fraction and/or insoluble chromatin. In some embodiments, the nuclei may be re-suspended and incubated in a low salt buffer to extract nucleoplasm-enriched proteins and/or euchromatin-associated proteins, and then centrifuged to facilitate collection of supernatant as a Nucleoplasm and Euchromatin Fraction and insoluble chromatin. In one embodiment, the nuclei may be re-suspended and incubated in a high salt buffer to extract nucleoplasm-enriched proteins, euchromatin-associated proteins and/or heterochromatin-associated proteins, and then centrifugated to facilitate collection of supernatant as a Nucleoplasm and Euchromatin Heterochromatin Fraction.

In some embodiments, after removal of the supernatant containing cytosolic proteins, the pellets are re-suspended in a sequential series of suspension solutions comprising increasing volatile salt concentrations, with each suspension solution used for selective extraction of various subsets of nuclear proteins, including nucleoplasm-enriched proteins, euchromatin-enriched proteins, and/or heterochromatin-enriched proteins. For example, (1) the nuclei may be re-suspended and incubated in an isotonic buffer to extract nucleoplasm-enriched proteins, followed by centrifugation to collect supernatant as a Nucleoplasm Fraction and insoluble chromatin; (2) the insoluble chromatin resulting from (1) may be re-suspended and incubated in a low salt buffer to extract euchromatin-associated proteins, followed by centrifugation to collect supernatant as a Euchromatin Fraction and insoluble chromatin; and (3) the insoluble chromatin resulting from (2) may be re-suspended and incubated in a high salt buffer to extract heterochromatin-associated proteins, following by centrifugation to collect supernatant as a Heterochromatin Fraction.

It is further understood that the samples do not necessarily have to be resuspended in each of the isotonic, low salt, and high salt buffers; instead; the method may be performed using one or two of these buffers, e.g., to selectively enrich for nuclear proteins associated with desired nuclear fraction(s). The nuclei and insoluble chromatin may be incubated in the various buffers for times and under conditions as described herein. For example, the nuclei and insoluble chromatin may be first incubated with an isotonic buffer to extract nucleoplasm-enriched proteins, followed by centrifugation to collect supernatant as a Nucleoplasm Fraction and insoluble chromatin, and the resulting insoluble chromatin may then be incubated in a high salt buffer to extract both euchromatin- and heterochromatin-associated proteins, followed by centrifugation to collect supernatant as a Euchromatin and Heterochromatin Fraction. For example, the nuclei and insoluble chromatin may be first incubated with a low salt buffer to extract nucleoplasm-enriched proteins and euchromatin-associated proteins, followed by centrifugation to collect supernatant as a Nucleoplasm and Euchromatin Fraction and insoluble chromatin, and the resulting insoluble chromatin may then be incubated in a high salt buffer to extract heterochromatin-associated proteins, followed by centrifugation to collect supernatant as a Heterochromatin Fraction.

The suspension solutions comprise one or more volatile salt. In some embodiments, the suspension solutions comprise one or more of the following volatile salts: ammonium acetate, ammonium bicarbonate, trimethylammonium acetate, or trimethylammonium bicarbonate. In some embodiments, the suspension solutions comprise ammonium acetate.

In some embodiments, the samples may be incubated in the suspension solution. In some embodiments, the samples may be incubated at a temperature of about 0° C., about 4° C., about 10° C., about 20° C., about 40° C., or about 100° C. The samples may be incubated at a temperature that is within a range defined by any two of the preceding values. In some embodiments, the samples may be incubated for a period of about 1 minute, about 3 minutes, about 5 minutes, about 10 minutes, about 20 minutes, about 30 minutes, about 50 minutes, or about 100 minutes. The samples may be incubated for a period that is within a range defined by any two of the preceding values.

In some embodiments, after removal of the supernatant containing cytosolic proteins, the remaining pellet is then resuspended in a second suspension buffer. In some embodiments, the second suspension buffer is an isotonic buffer comprising one or more volatile salt. In some embodiments, the second suspension buffer is an isotonic buffer comprising one or more of the following volatile salts: ammonium acetate, ammonium bicarbonate, trimethylammonium acetate, or trimethylammonium bicarbonate. In some embodiments, the second suspension buffer is an isotonic buffer comprising ammonium acetate. In some embodiments, an isotonic buffer has approximately physiological salt concentrations. In some embodiments, an isotonic buffer comprises a volatile salt concentration of about 10 mM to about 150 mM. In some embodiments, an isotonic buffer comprises an ammonium acetate concentration of about 10 mM to about 150 mM. In some embodiments, an isotonic buffer comprises a volatile salt concentration, e.g., an ammonium acetate concentration, of about 5 mM, about 10 mM, about 15 mM, about 20 mM, about 25 mM, about 50 mM, about 75 mM, about 100 mM, about 125 mM, or about 150 mM. In some embodiments, an isotonic buffer comprises an ammonium acetate concentration of about 15 mM. In some embodiment, the second suspension buffer is an isotonic buffer comprising 10 mM Tris pH 8.0, 60 mM KCl, 1.5 mM EDTA, and 15 mM ammonium acetate.

In some embodiments, the sample resuspended in the second suspension buffer is incubated at 4° C. for 15 minutes. The samples are then centrifuged. In some embodiments, the samples are centrifuged at 500 RCF for 5 minutes at 4° C. The supernatant is then removed. In some embodiments, this supernatant can be discarded. In some embodiments, this supernatant can be retained as the Nucleoplasm Fraction.

In some embodiments, after removal of the supernatant containing the nucleoplasm fraction, the remaining pellet is then resuspended in a third suspension buffer. In some embodiments, the third suspension buffer is a low salt buffer comprising one or more volatile salt. In some embodiments, the third suspension buffer is a low salt buffer comprising one or more of the following volatile salts: ammonium acetate, ammonium bicarbonate, trimethylammonium acetate, or trimethylammonium bicarbonate. In some embodiments, the third suspension buffer is a low salt buffer comprising ammonium acetate. In some embodiments, the third suspension buffer is a low salt buffer comprising a volatile salt concentration of about 200 mM to about 500 mM. In some embodiments, the third suspension buffer is a low salt buffer comprising a volatile salt concentration, e.g., an ammonium acetate concentration, of about 200 mM, about 225 mM, about 250 mM, about 275 mM, about 300 mM, about 350 mM, about 400 mM, about 450 mM, or about 500 mM. In some embodiments, the third suspension buffer is a low salt buffer comprising an ammonium acetate concentration of about 250 mM. In some embodiments, the third suspension buffer is a low salt buffer comprising 10 mM Tris pH 8.0, 1.5 mM EDTA, and 250 mM ammonium acetate.

In some embodiments, the sample resuspended in the third suspension buffer is incubated at 4° C. for 15 minutes. The samples are then centrifuged. In some embodiments, the samples are centrifuged at 1,000 RCF for 5 minutes at 4° C. The supernatant is then removed. In some embodiments, this supernatant can be discarded. In some embodiments, this supernatant can be retained as the Euchromatin Fraction.

In some embodiments, after removal of the supernatant containing the euchromatin fraction, the remaining pellet is then resuspended in a fourth suspension buffer. In some embodiments, the fourth suspension buffer is a high salt buffer comprising one or more volatile salt. In some embodiments, the fourth suspension buffer is a high salt buffer comprising one or more of the following volatile salts: ammonium acetate, ammonium bicarbonate, trimethylammonium acetate, or trimethylammonium bicarbonate. In some embodiments, the fourth suspension buffer is a high salt buffer comprising ammonium acetate. In some embodiments, the fourth suspension buffer is a high salt buffer comprising a volatile salt concentration of greater than about 500 mM, e.g., about 500 mM to about 1,000 mM or about 550 mM to about 1000 mM. In some embodiments, the fourth suspension buffer is a high salt buffer comprising a volatile salt concentration, e.g., an ammonium acetate concentration, of about 550 mM, about 575 mM, about 600 mM, about 650 mM, about 700 mM, about 750 mM, about 800 mM, about 900 mM, or about 1000 mM. In some embodiments, the fourth suspension buffer is a high salt buffer comprising an ammonium acetate concentration of about 600 mM. In some embodiments, the fourth suspension buffer is a high salt buffer comprising 10 mM Tris pH 8.0, 1.0 mM EDTA, and 600 mM ammonium acetate.

In some embodiments, the sample resuspended in the fourth suspension buffer is incubated at 4° C. for 15 minutes. The samples are then centrifuged. In some embodiments, the samples are centrifuged at 20,000 RCF for 5 minutes at 4° C. The supernatant is then removed. In some embodiments, this supernatant can be discarded. In some embodiments, this supernatant can be retained as the Heterochromatin Fraction. The remaining pellet contains the insoluble fraction and may be stored for future use.

### Cell Nuclei Isolation and Protein Extraction Using Lectin-Coupled Solid Supports and Volatile Salt-Based Buffers and Solutions

In particular embodiments of the methods of extracting proteins disclosed herein, the nuclei are isolated and/or manipulated through the use of lectins that bind to nuclear glycans. In particular embodiments, the nuclei are bound to a lectin-coupled solid support. In certain embodiments, nuclei bound to a lectin-coupled solid support are contacted with one or more of the volatile salt-containing buffers disclosed herein to extract nuclear and/or chromatin-associated proteins from the nuclei. In particular embodiments, one or more of nucleoplasm-associated proteins, euchromatin-associated proteins, and/or heterochromatin-associated proteins are extracted from nuclei bound to lectins coupled to a solid support.

### Lectins and Glycans

Lectins are a class of proteins that bind to certain sugar groups, or glycans. Concanavalin A (ConA), a lectin that binds selectively to an N-glycan called mannose, has been shown through staining to bind to the nuclear membrane (see Monneron et. al. Extensive Binding of Concanavalin A to the Nuclear Membrane. FEBS Lett. 1974, 42 (2), 209-213). However, the evidence of N-glycans actually occurring on the surface of the nuclear membrane is limited (see Stanley et. al. N-Glycans; Cold Spring Harbor Laboratory Press, 2022).

The present disclosure provides data showing that mannoses are enriched on the outer cell membrane and not the nuclear membrane. The nuclear membrane is instead enriched with glycoproteins containing glycans such as N-acetyllactosamine (LacNAc), galactose, N-acetylgalactosamine (GalNAc), and N-acetylglucosamine (GlcNAc). Erythrina crista-galli lectin (ECL) and Ricinus communis agglutinin (RCA), e.g., RCA120, are lectins that bind to galactose, N-acetylgalactosamine (GalNAc) and N-acetyllactosamine (LacNAc). ECL and RCA, e.g., RCA120, are superior in specificity and binding capability as compared to ConA in binding, isolating, or enriching cell nuclei, thereby reducing loss of cell nuclei during sample processing and increasing purity of isolated cell nuclei or fractions or components thereof when used in the methods described herein. Additionally, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin also have superior binding specificity for N-acetyllactosamine (LacNAc), galactose, N-acetylgalactosamine (GalNAc), and/or N-acetylglucosamine (GlcNAc) and can also be used in the methods described herein.

Lectins, such as ECL and RCA, that bind to glycans enriched on the nuclear membrane may be coupled to a solid support to facilitate handling, isolating, and/or extracting cell nuclei and subfractions thereof. In some embodiments, the one or more lectin coupled to the solid support comprises Erythrina crista-galli lectin (ECL), Ricinus communis agglutinin (RCA), Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectins comprise both Erythrina crista-galli lectin and Ricinus communis agglutinin. In some embodiments, the lectin is Erythrina crista-galli lectin. In some embodiments, the lectin is Ricinus communis agglutinin (RCA). In some embodiments, the Ricinus communis agglutinin is RCA120. In some embodiments, the one or more glycan is enriched on the nuclear membrane. In some embodiments, the one or more glycan comprises one or more of N-acetyllactosamine (LacNAc), galactose, N-acetylgalactosamine (GalNAc), and/or N-acetylglucosamine (GlcNAc). In some embodiments, the one or more glycans is a combination of any of the glycans selected from a list consisting of N-acetyllactosamine (LacNAc), galactose, N-acetylgalactosamine (GalNAc), and/or N-acetylglucosamine (GlcNAc).

### Lectin-Coupled Solid Support

As described herein, a “solid support” comprises any flexible or rigid substrate or material onto which one or more binding agent is applied. In some embodiments, the solid support is a planar surface. In some embodiments, the solid support has a tubular surface. In some embodiments, the solid support has a circular surface. In some embodiments, the solid support is a bead. In some embodiments, the solid support is magnetic. In some embodiments, the solid support is a magnetic bead. In some embodiment, the solid support is biocompatible, wherein it is not toxic to living cells or tissues. In some embodiments, the surface supporting the one or more binding agent comprises a material or reagent different from that of the rest of the substrate. In some embodiments, the solid support has a functionalized surface for coupling to proteins. In some embodiments, the solid support has a functionalized surface for coupling to lectins. In some embodiments, the solid support has a surface comprising streptavidin (streptavidin-coated). In some embodiments, the one or more lectin binds to one or more glycan enriched on the nuclear membrane. In some embodiments, one or more lectin that binds to N-acetyllactosamine (LacNAc), galactose, N-acetylgalactosamine (GalNAc), and/or N-acetylglucosamine (GlcNAc) is coupled to a surface of the solid support. In some embodiments, the one or more lectins are coupled to the surface of the solid support via a biotin-streptavidin conjugation. In some embodiments, the one or more lectin is biotinylated and binds to the surface of a solid support that is streptavidin-coated via biotin-streptavidin conjugation. In some embodiments, the one or more lectin comprises Erythrina crista-galli lectin (ECL), Ricinus communis agglutinin (RCA), e.g., RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectin is Erythrina crista-galli lectin (ECL). In some embodiments, the lectin is Ricinus communis agglutinin (RCA).

In some embodiments, a sample comprising nuclei isolated from lysed cells is added to and/or mixed with a lectin-coupled solid support, so the nuclei become associated with the solid support via the lectin and then further exposed to buffers comprising one or more different concentrations of volatile support to extract selected nuclear and/or chromatin-associated proteins.

In some embodiments, the sample resuspended in the second suspension buffer comprising a volatile salt as described above is then added to and/or mixed with a lectin-coupled solid support. For example, activated lectin-conjugated magnetic beads may be added to the sample with the pellet and the isotonic buffer and mixed by pipetting up and down. In some embodiment, activated lectin-conjugated magnetic beads are added at a volume of 20 μl per million cells.

The samples are then contacted with the lectin-coupled solid support for a time sufficient for the cell nuclei to bind to the lectin-coupled solid support. In some embodiments, the contact period is at least 5, 10, or 20 minutes. In some embodiments, the sample is subjected to gentle shaking during the contact period. In some embodiments, the sample is placed on wet ice or kept at 4° C. during the contact period. After the cell nuclei are bound to the solid support, the supernatant is separated from the cell nuclei bound to the solid support through centrifugation or other means known in the arts. For example, cell nuclei bound to beads can be centrifuged. In some embodiments, cell nuclei bound to lectin-conjugated magnetic beads are placed onto a magnetic rack and allowed to sit for a sufficient amount of time for the magnetic beads to pellet. The supernatant is then removed carefully and retained as the Nucleoplasm Fraction.

In some embodiments, the sample containing the pellet is then resuspended in a third suspension buffer comprising a volatile salt. In some embodiments, the third suspension buffer is a low salt buffer comprising one or more volatile salt. In some embodiments, the third suspension buffer is a low salt buffer comprising one or more of the following volatile salts: ammonium acetate, ammonium bicarbonate, trimethylammonium acetate, or trimethylammonium bicarbonate. In some embodiments, the third suspension buffer is a low salt buffer comprising ammonium acetate. In some embodiments, the third suspension buffer is a low salt buffer comprising a volatile salt concentration of about 200 mM to about 500 mM. In some embodiments, the third suspension buffer is a low salt buffer comprising an ammonium acetate concentration of about 250 mM. In some embodiments, the third suspension buffer is a low salt buffer comprising 10 mM Tris pH 8.0, 1.5 mM EDTA, and 250 mM ammonium acetate. In some embodiments, the resuspended pellet is incubated in the third suspension buffer at 4° C. for 15 minutes. The supernatant is then separated from the pellet containing cell nuclei, insoluble chromatin and/or insoluble proteins bound to the solid support through centrifugation or other means known in the arts as described above. For example, cell nuclei and/or insoluble chromatin bound to lectin-conjugated magnetic beads are placed onto a magnetic rack and allowed to sit for a sufficient amount of time for the magnetic beads to pellet. The supernatant is then removed carefully and retained as the Euchromatin Fraction.

In some embodiments, the sample containing the pellet is then resuspended in a fourth suspension buffer comprising a volatile salt. In some embodiments, the fourth suspension buffer is a high salt buffer comprising one or more volatile salt. In some embodiments, the fourth suspension buffer is a high salt buffer comprising one or more of the following volatile salts: ammonium acetate, ammonium bicarbonate, trimethylammonium acetate, or trimethylammonium bicarbonate. In some embodiments, the fourth suspension buffer is a high salt buffer comprising ammonium acetate. In some embodiments, the fourth suspension buffer is a high salt buffer comprising a volatile salt concentration of about 500 mM to about 1,000 mM. In some embodiments, the fourth suspension buffer is a high salt buffer comprising an ammonium acetate concentration of about 600 mM. In some embodiments, the fourth suspension buffer is a high salt buffer comprising 10 mM Tris pH 8.0, 1.0 mM EDTA, and 600 mM ammonium acetate. In some embodiments, the resuspended pellet is incubated in the fourth suspension buffer at 4° C. for 15 minutes. The supernatant is then separated from the pellet containing cell nuclei and/or insoluble chromatin bound to the solid support through centrifugation or other means known in the arts as described above. For example, cell nuclei and/or insoluble chromatin bound to lectin-conjugated magnetic beads are placed onto a magnetic rack and allowed to sit for a sufficient amount of time for the magnetic beads to pellet. The supernatant is then removed carefully and retained as the Heterochromatin Fraction. The remaining pellet contains the insoluble fraction and may be stored for future use.

In some embodiments, nuclear proteins etc. may be extracted using a single buffer, e.g., a high salt buffer. In some embodiments, nuclear proteins etc. may be extracted using two or more buffers consecutively, e.g., an isotonic buffer and a high salt buffer, or a low salt buffer and a high salt buffer.

In some embodiments, cell nuclei are resuspended in a high salt buffer comprising a volatile salt after isolation with the lectin-coupled solid support to extract chromatin-associated proteins. In some related embodiments, the isotonic buffer is not used to separate the nucleoplasm fraction prior to resuspension in the high salt buffer comprising a volatile salt.

In some embodiments, a high salt buffer comprising a volatile salt can be used to extract a chromatin fraction comprising chromatin-associated proteins. For example, after cell nuclei are isolated with the lectin-coupled solid support and a Nucleoplasm Fraction is collected, as described previously, the pellet is then resuspended into the high salt buffer. In some embodiments, the resuspended pellet is incubated in the high salt buffer at 4° C. for 15 minutes. The supernatant is then separated from the pellet containing cell nuclei and/or insoluble chromatin bound to the solid support through centrifugation or other means known in the arts as described above. For example, cell nuclei and/or insoluble chromatin bound to lectin-conjugated magnetic beads are placed onto a magnetic rack and allowed to sit for a sufficient amount of time for the magnetic beads to pellet. The supernatant is then removed carefully and retained as the Chromatin Fraction. The remaining pellet contains the insoluble fraction and may be stored for future use.

### High-Throughput Processing

High-throughput methods are often more cost-effective and increase efficiency when a large number of samples need to be analyzed. The use of volatile salt buffers for protein extraction described herein can be carried out in any compatible high-throughput format, such as with the use of multi-well plates and lectin-coupled solid supports. For example, multiple samples coupled to lectin-conjugated beads may be placed within multiple wells of a 96-well plate to be centrifuged and washed. In some embodiments, the lectin-conjugated beads are magnetic and a magnet compatible with the multi-well plate is used to collect or bind the magnetic beads in order to pellet, move, or transfer the sample. In some embodiments, the magnet immobilizes the samples bound to the magnetic beads while the supernatant is removed or while a suspension buffer comprising one or more volatile salt is added. In some embodiments, the magnet binds to the samples bound to the magnetic beads and move or transfer the samples into a vessel, such as a separate multi-well plate. In some embodiments, an automation instrument may be used to process samples as described herein. In some embodiments, the instrument can be an automated liquid handler. In some embodiment, the instrument can be a magnetic bead handling robot. In some embodiments, the instrument can be an automated sample purification or extraction system, such as the Thermo Scientific KingFisher Purification System. In some embodiments, one or more of the steps to isolate or extract protein fractions can be performed using the automated system. In some embodiments, all the isolation and extraction steps are performed using the automated system.

In some embodiments, the high throughput method comprises multiple samples which are present in a buffer within multiple wells of a sample plate and a solid support of magnetic beads, wherein the cell nuclei bound to the solid support are removed from the multiple wells using magnets. In some embodiments, the cell nuclei bound to the solid support are then placed into a second buffer comprising one or more volatile salts within multiple wells of a sample plate, wherein the second buffer may be the same or a different buffer from the preceding buffer. In some embodiments, the steps of removing the sample bound to the solid support from the multiple wells using magnets and placing into a second buffer may be repeated one or more times. In some embodiments, the sample bound to the solid support, wherein the solid support is lectin-conjugated magnetic beads, are removed from the multiple wells using magnets and sequentially placed into and removed from a series of buffers, wherein the buffers comprise an isotonic buffer to extract nucleoplasm proteins, a low salt buffer to extract euchromatic proteins, and a high salt buffer to extract heterochromatin proteins, wherein the buffers comprise one or more volatile salt.

In particular embodiments, any of the methods disclosed herein comprises one or more of: (1) using detergents (NP-40) for nuclear isolation to ensure that the proteins are not denatured during the process; (2) quenching with EDTA and/or use of protease inhibitors to prevent protein degradation during the process; (3) not using buffers with polycations, which interferes with protein analysis; and (4) sonication or removal of DNA/RNA by enzymatic processes.

### Sample Analysis

Nuclei isolated and/or manipulated via the use of volatile salts may be analyzed via a variety of means. For example, they may be treated with different agents or under various biological or environmental conditions, and the effect on the nuclei examined. In some embodiments, the effect on the amount or localization of various proteins within the nuclei is determined. Protein amounts and/or identity within nuclei or nuclear protein fractions may be analyzed via a variety of means known in the art, including but not limited to, mass spectrometry and/or immunological methods, such as western blotting and other methods using antibodies specific for nuclear proteins of interest.

In some embodiments, after one or more of the Cytoplasmic Fraction, Nuclear Fraction, Nucleoplasm Fraction, Chromatin Fraction, Euchromatin Fraction, and/or Heterochromatin Fraction are obtained as described herein, they may be analyzed via a variety of means known in the art, e.g., to determine the identity of the proteins within the fraction(s). In particular embodiments, they are analyzed via mass spectrometry to determine the presence and/or absence of peptides and proteins present in the samples.

In some embodiments, proteins in the sample(s) are digested using trypsin protease, following by mass spectrometry analysis. Mass spectrometry data may be collected using a data-dependent acquisition strategy and peptides modified by the electrophilic label, e.g., NM-IAA, are identified based on the detection of the mass modification of NM-IAA (about 71.03711 Da) as a variable modification on cysteine residues using any proteomics search software.

In particular embodiments, samples may be analyzed using mass spectrometry (MS), such as tandem mass spectrometry (MS-MS), time-of-flight mass spectrometry (TOF-MS), quadrupole mass spectrometry (Q-MS), ion trap IT-MS, orbitrap mass spectrometry, or any combination thereof. The samples may be analyzed using a combination of chromatographic and mass spectrometric techniques, such as gas chromatography-mass spectrometry (GC-MS), liquid chromatography-mass spectrometry (LC-MS), or high-pressure liquid chromatography-mass spectrometry (HPLC-MS).

### Applications

The methods disclosed herein may be used for a variety of purposes. For example, the methods disclosed herein may be used to compare nuclear proteomes across two or more conditions. For instance, the methods described herein may be used to characterize nuclear proteome changes in response to one or more external or internal perturbations. Such perturbations may include, but are not limited to, a change in cell state (e.g., cell cycle), cell environment, or exposure of the cell to a chemical treatment or physical stress. Such changes may be detected by changes in the location of proteins, e.g., proteins moving from one or more of the cytoplasm, nucleoplasm, euchromatin, or heterochromatin. For example, the methods may be used to identify functional systems or proteins, e.g., transcription factors (TFs), with alterations associated with an external or internal perturbations, such as alterations in cellular location.

In some embodiments, the methods may be used to diagnose a disease or disorder, including any of those described herein, including but not limited to, cancer, infection, immunological disease, metabolic disease, cardiac disease, inflammatory disease, etc. For example, a cell sample obtained from a subject may be analyzed as described herein, and results related to the location of cellular proteins (e.g., TFs or kinases) can be compared to the results obtained from healthy cells and/or diseased cells, or a predetermined set of results from healthy cells and/or diseased cells, thereby determining whether the cells are healthy or diseased. In some embodiments, the methods are used to identify cytosolic and/or nuclear proteins, e.g., TFs, having a change in location in diseased cells as compared to healthy cells. In some embodiments, the methods are used to identify and/or analyze transcription factors, kinases, and/or proteins involved in nuclear import/trafficking.

In some embodiments, the methods disclosed herein may be used to characterize small molecule degradation compounds. The methods described herein, such as methods 100, 200, or 300, may be used to screen small molecule degraders in an unbiased manner to identify proteins targeted for degradation in response to treatment.

In some embodiments, the methods disclosed herein may be used to assay cellular thermal shifts. The assay may detect compound engagement with the protein target in living cells by measuring changes in thermal stability of the protein. The methods described herein may be used to profile thermal stability of the nuclear proteome and to study compounds interacting with nuclear proteins.

In some embodiments, the methods disclosed herein may be used to characterize genome edits. Genome editing with clustered regularly interspersed palindromic repeats (CRISPR)-based genome editing techniques, transcription activator-like effector nuclear (TALEN)-based genome editing techniques, zinc finger-based genome editing techniques, or other nuclease technologies can create mutations in the DNA of a cell. These mutations may result in global changes in the proteome and/or the nuclear proteome which may be detected with the methods described herein.

The use of lectins bound to solid surfaces provides the ability to process, manipulate, process, and/or analyze the nuclei for a variety of different applications. For example, nuclei bound to lectins bound to the solid surface may be treated to one or more different conditions, and then isolated via the solid surface, e.g., to examine biological effects of the one or more different conditions. In particular embodiments, having the nuclei bound to the solid support via the lectins allows for easy removal of liquids, e.g., containing various active molecules being tested, from the nuclei, e.g., by removing the liquid from the support-bound nuclei, or by removing the support-bound nuclei from the liquid. In particular embodiments, the nuclei may be subjected to a series of different conditions by moving the nuclei from one condition to another via the solid support to which they are bound. This may be done, e.g., using lectins bound to a magnetic bead, which can then be removed with associated nuclei using a magnet.

In some embodiments, the methods disclosed herein may be used to enable single cell analysis, e.g., where cell counts are low, including but not limited to handling or manipulating nucleus from a single cell or from tens or hundreds of nuclei. Use of the methods disclosed herein to handle low numbers of nuclei is useful for single cell proteomics, genomics, and transcriptomics.

In some embodiments, the methods disclosed herein may be used to isolate nuclei from tissues, including, e.g., previously fixed tissues, including but not limited to Formalin-fixed, Paraffin-Embedded (FFPE) tissues, paraformaldehyde (PFA) fixed tissues, or alcohol (e.g., ethanol or methanol) fixed tissues. Similarly, the methods mat be used to remove formalin, paraffin, or FDA from isolated nuclei. In some embodiments, the nuclei isolated from fixed tissues can be analyzed by flow cytometry or used for fluorescence-activated cell sorting (FACS).

### Definitions

Unless defined otherwise, all technical and scientific terms used herein have the same meaning as is commonly understood by one skilled in the arts to which the claimed subject matter belongs. It is to be understood that the foregoing and the following descriptions are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise.

In this application, the use of “or” means “and/or” unless stated otherwise. Furthermore, use of the term “including” as well as other forms, such as “include”, “includes,” and “included,” is not limiting.

As used herein, ranges and amounts may be expressed as “about” a particular value or range. About also includes the exact amount. Hence “about 5 μL” means “about 5 μL” and also “5 L.” Generally, the term “about” includes an amount that would be expected to be within experimental error. As used in this application, the terms “about” and “approximately” are used as equivalents. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used herein, the terms “lectin-coupled solid support” or “lectin-conjugated solid support” are used interchangeably and refer to a solid support, wherein one or more lectins is on the surface of the solid support. The one or more lectin can be attached to the solid support as described in the present disclosure or through any means known in the arts.

### EXAMPLES

### Example 1: Extraction of Protein Fractions Using Buffers Containing Volatile Salts

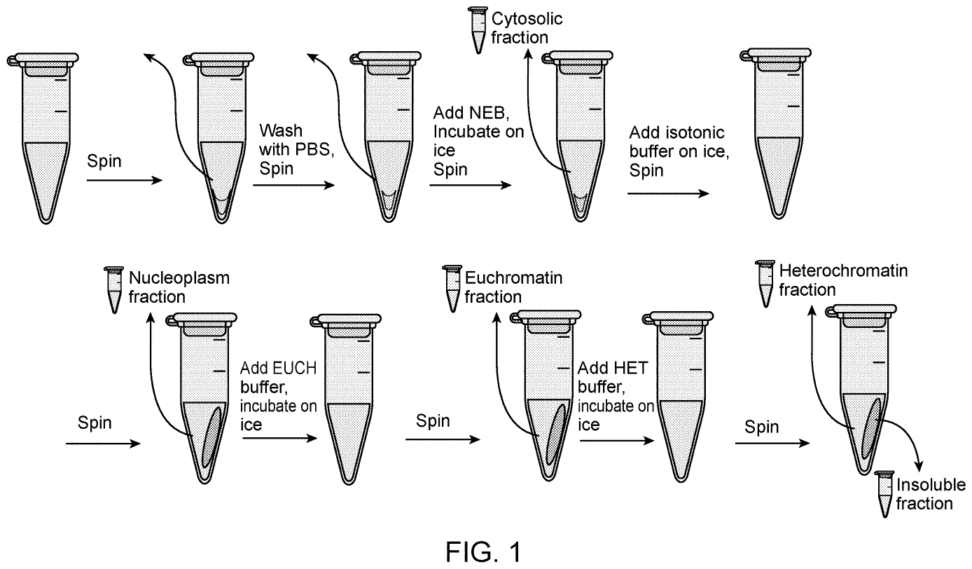

An exemplary method for isolating four protein fractions (cytosolic, euchromatin, heterochromatin, and nucleoplasm) using buffers containing volatile salts is illustrated in FIG. 1. Formulations of buffers used for the isolation of these protein fractions can be found in Table 1.

TABLE 1
Volatile salt extraction buffers

| | | Concentration | |
|---|---|---|---|
| | Component | (mM or %) | |
| Nuclear Extraction | HEPES | 20 | mM |
| Buffer 1 | KCl | 10 | mM |

| 10% Triton-X 100 | 0.1% | |
|---|---|---|
| Glycerol | 20 | mM |
| MnCl2 | 1 | mM |

| | | Concentration |
|---|---|---|
| | Component | (mM) |
| Ammonium Acetate | Tris | 10 |
| (AA) Isotonic Buffer | Ammonium Acetate (AA) | 15 |
| | KCl | 60 |
| | EDTA | 1.5 |
| AA Euchromatin | Tris | 10 |
| Buffer | Ammonium Acetate (AA) | 250 |
| | EDTA | 1.5 |
| AA Heterochromatin | Tris | 10 |
| Buffer | Ammonium Acetate (AA) | 600 |
| | EDTA | 1 |

As shown in FIG. 1, to isolate the cytosolic fraction, nucleoplasm fraction, euchromatin fraction, and heterochromatin fraction, cells were collected into 1.5 ml polypropylene tubes and centrifuged at 400 RCF for 5 minutes at 4° C. The supernatant was removed and discarded. Then the cell pellets were washed with 1 mL each of ice cold 1×PBS by thorough resuspending and centrifuged at 400 RCF for 5 minutes at 4° C. The supernatant was removed and discarded. Then each pellet was resuspended into 200 μl of Nuclear Extraction Buffer 1 with a clean pipette tip by pipetting up and down at least 3 times. The samples were incubated for 10 minutes on a cold block or wet ice. After the incubation, the samples were centrifuged at 400 RCF for 5 minutes at 4° C. The supernatant was removed, transferred to a clean Eppendorf tube, and retained as the Cytosolic Fraction.

The pellets containing cell nuclei were then resuspended in 200 μl of AA Isotonic Buffer by gently pipetting up and down. The samples were then incubated at 4° C. for 15 minutes. After the incubation, the samples were centrifuged at 500 RCF for 5 minutes at 4° C. The supernatant was carefully removed, transferred to a clean Eppendorf tube, and retained as the Nucleoplasm Fraction.

The pellets were then resuspended in 200 μl of AA Euchromatin Buffer and incubated at 4° C. for 15 minutes. After the incubation, the samples were centrifuged at 1,000 RCF for 5 minutes at 4° C. The supernatant was carefully removed, transferred to a clean Eppendorf tube, and retained as the Euchromatin Fraction.

The pellets were then resuspended in 200 μl of AA Heterochromatin Buffer and incubated at 4° C. for 15 minutes. After the incubation, the samples were centrifuged at 20,000 RCF for 5 minutes at 4° C. The supernatant was carefully removed, transferred to a clean Eppendorf tube, and retained as the Heterochromatin Fraction. The remaining pellet contained the insoluble fraction and could be stored for future use.

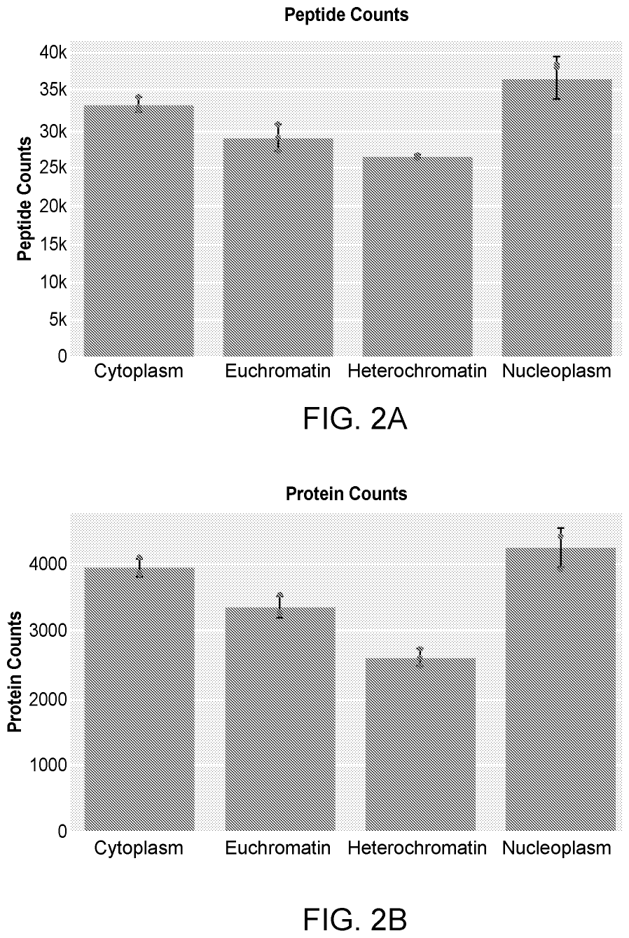

To assess the compatibility of volatile salt-based buffers with LC-MS, the cytosolic fraction, nucleoplasm fraction, euchromatin fraction, and heterochromatin fraction that were extracted from 5 million THP.1 cells using ammonium acetate-based buffers were analyzed by LC-MS. As shown in FIG. 2A, the average peptide counts across triplicate measurements (three separate samples each with a unique MS run) were approximately 32,813 in the cytoplasm fraction, approximately 28,813 in the euchromatin fraction, approximately 26,367 in the heterochromatin fraction, and approximately 36,620 in the nucleoplasm fraction. As shown in FIG. 2B, the protein counts were approximately 3949 in the cytoplasm fraction, approximately 3,361 in the euchromatin fraction, approximately 2,602 in the heterochromatin fraction, and approximately 4,250 in the nucleoplasm fraction. These peptide and protein counts demonstrate that samples extracted in volatile salt-based buffers can be analyzed by LC-MS without sample clean-up steps involving buffer exchange as there is a comparable number of peptides and proteins detected in these samples compared to conventional sample preparations that use non-volatile salts.

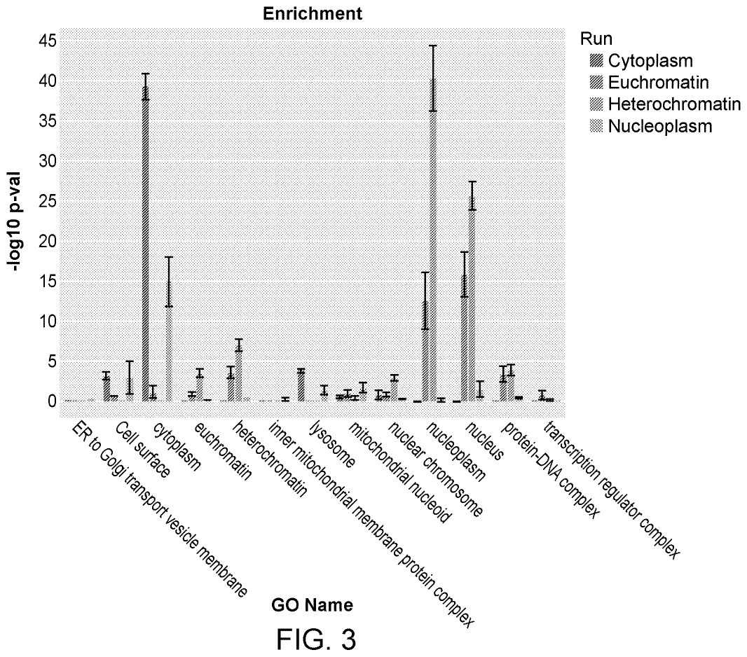

Additionally, the proteins found in the four protein fractions (cytoplasm, euchromatin, heterochromatin, and nucleoplasm) isolated using buffers containing ammonium acetate and analyzed by LC-MS were subjected to GO (gene ontology) enrichment analysis. As shown in FIG. 3, each ammonium acetate-based buffer extracts proteins that match the expected protein groups for each fraction.

### Example 2: Comparison of the Use of Ammonium Acetate-Based Buffers and Sodium Chloride-Based Buffers to Extract Protein Fractions

To determine whether the use of ammonium acetate-based buffers to extract proteins yield comparable results as the use of sodium chloride-based buffer, the cytoplasm, nucleoplasm, euchromatin, and heterochromatin fractions were extracted from 5 million THP.1 cells separately with both buffers. The samples were further processed with a desalting/washing step. Briefly, proteins from the samples were aggregated onto carboxyl-coated magnetic beads using 50% ethanol. The beads, coated in proteins, were washed 5 times in 80% ethanol to remove salts and other impurities, and then the beads were resuspended in aqueous ammonium bicarbonate buffer. The proteins were digested into peptides using trypsin protease. All samples were then analyzed by LC-MS. The formulations of the ammonium acetate (AA)-buffers used in the experiment are provided in Table 1, and the sodium chloride (NaCl)-based buffers used in the experiment are provided in Table 2.

TABLE 2
Non-volatile salt extraction buffers

| Buffer | Components and concentrations |
|---|---|
| Nuclear Extraction Buffer 2 | 20 mM HEPES, 10 mM KCl, |
| | 20% Glycerol, 1 mM MnCl₂, |
| | 0.1% Triton X-100, |
| | 0.5 mM spermidine |
| NaCl Isotonic Buffer | 10 mM Tris pH 8.0, 15 mM NaCl, |
| | 60 mM KCl, 1.5 mM EDTA pH 8.0 |
| NaCl Euchromatin Buffer | 10 mM Tris pH 8.0, 250 mM NaCl, |
| | 1 mM EDTA pH 8.0 |
| NaCl Heterochromatin Buffer | 10 mM Tris pH 8.0, 600 mM NaCl, |
| | 1 mM EDTA pH 8.0 |

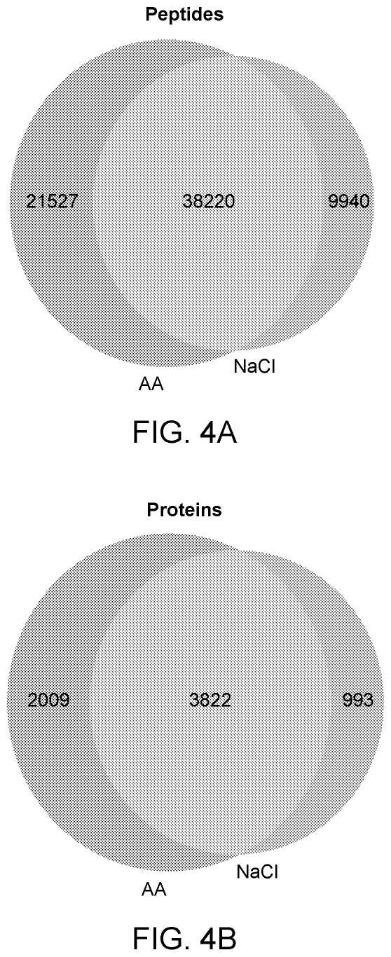

After LC-MS analysis, the number of peptides and proteins that overlapped in the samples was determined. As shown in the Venn diagram in FIG. 4A, of the 59,747 peptides identified in the sample extracted using ammonium acetate-based buffers, 38,200 overlapped with those identified in the sample extracted using NaCl-based buffers while 21,527 peptides were unique to the ammonium acetate sample and 9,940 peptides were unique to the NaCl sample. As shown in FIG. 4B, of the 5,831 proteins identified in the sample extracted using ammonium acetate-based buffers, 3,822 overlapped with those identified in the sample extracted using NaCl-based buffers while 2,009 proteins were unique to the ammonium acetate sample and 993 proteins were unique to the NaCl sample. These results demonstrate that ammonium acetate-based buffers perform similarly, if not better, than NaCl-based buffers in the extraction of peptides and proteins from the cytoplasm, nucleoplasm, euchromatin, and heterochromatin fractions. Notably, more total peptides and proteins were identified in the samples extracted using ammonium acetate-based buffers compared to those extracted using NaCl-based buffers. The use of acetate-based buffers for extraction, which eliminates the need for a desalting step, led to increased numbers of peptides and proteins identified by LC-MS.

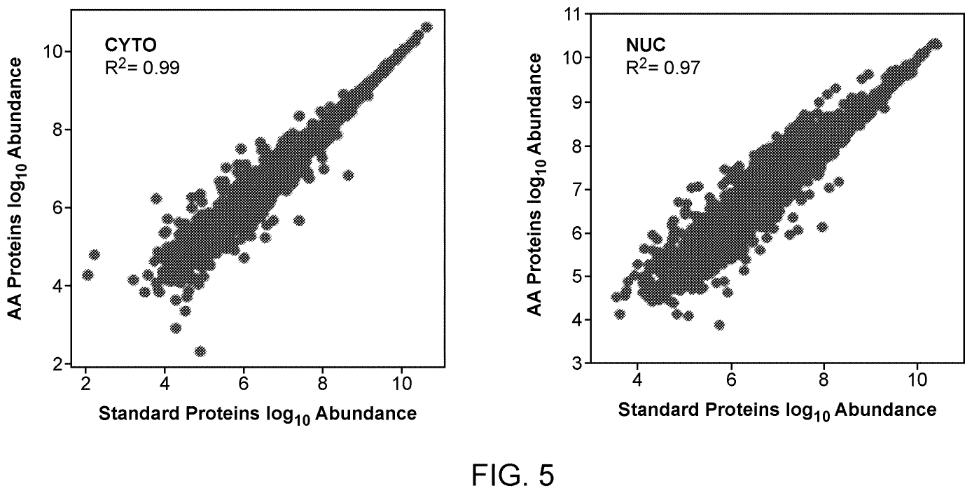

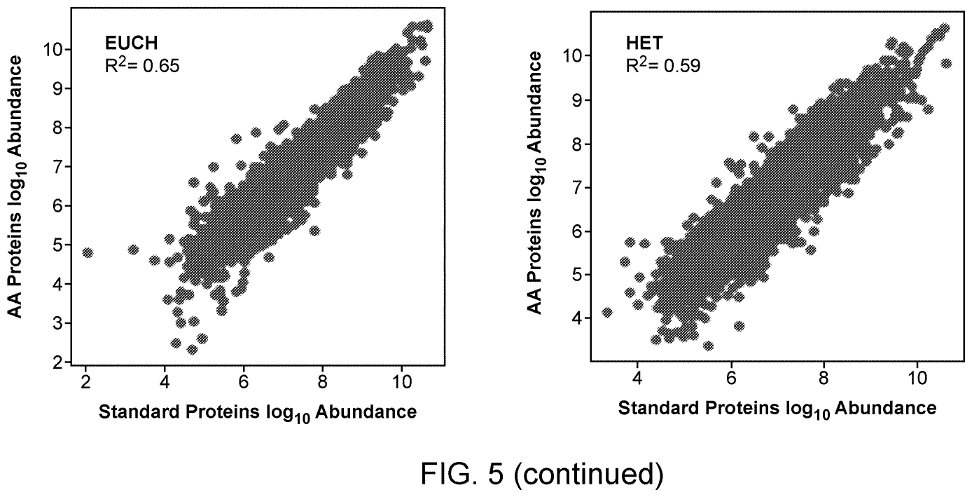

Additionally, protein abundances of proteins identified from the LC-MS analysis were determined for samples extracted using ammonium acetate (AA)-based extraction buffers and for samples extracted using NaCl-based extraction buffers. The Pearson correlation coefficient for the protein abundances of each of the two buffers (ammonium acetate on the y-axis and NaCl on the x-axis) was calculated for each protein fraction: cytoplasm, nucleoplasm, euchromatin, and heterochromatin. As shown in FIG. 5, the correlation coefficient (r) values for each of the cytoplasm, nucleoplasm, euchromatin, and heterochromatin fractions were 0.99, 0.98, 0.81, and 0.77, respectively. The coefficient of determination (r2) values were 0.99, 0.97, 0.65, and 0.59, respectively. Together, these results show that protein abundances as determined by MS is highly correlated when use of volatile salt-based extraction buffers are compared to conventional non-volatile salt-based extraction buffers when used for protein extraction and subsequent MS analysis.

### Example 3: Identification of Lectins that Bind to Glycans Enriched on the Nuclear Membrane

To determine glycans that are enriched on the nuclear membrane, 5 million MM1. S cells were subjected to chromatin extraction, and the cytosolic (containing the outer cell membrane) and insoluble (containing the nuclear membrane) fractions were analyzed using glycomic mass spectrometry. Gal indicates D-galactose; GlcNAc indicates N-acetylglucosamine; Man indicates D-mannose; Glc indicates D-glucose; and GalNAc indicates N-acetylgalactosamine. Paucimannose glycans include relatively simple mannose (Man) and N-acetylglucosamine (GlcNAc)-containing glycans.

TABLE 3
Lectin and glycan pairs that bind with specificity

| Lectin | Glycan epitope names |
|---|---|
| Amaranthus caudatus lectin | Galb1-3GalNAc, LacNAc |
| Datura Stramonium lectin | LacNAc, GlcNAcb1-4GlcNAc |
| Erythrina crista-galli lectin | LacNAc, galactose, GalNAc |
| Lycopersicon esculentum lectin | LacNAc |
| Maackia amurensis agglutinin I | 3′sulfo-LacNAc, 3′sulfo-Lac |
| Ricinus communis agglutinin I | LacNAc, galactose, GalNAc |

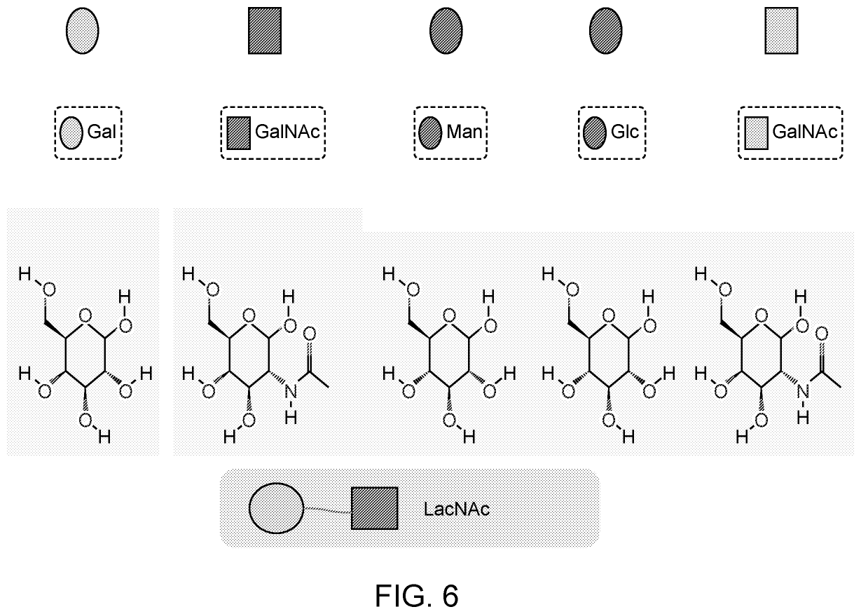

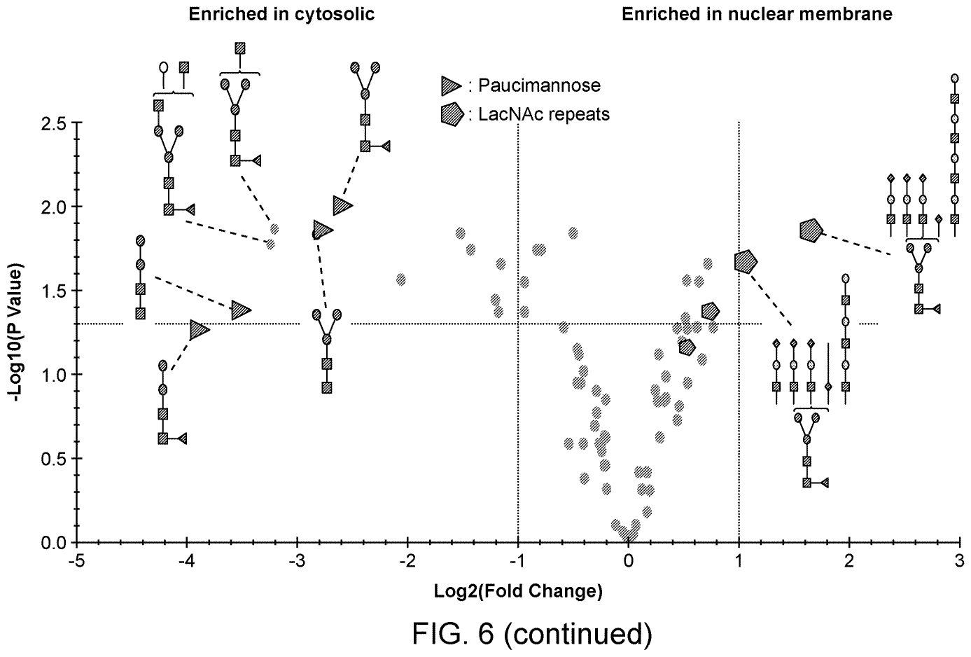

As shown in the volcano plot in FIG. 6, the cytosolic fraction was enriched with glycoproteins containing paucimannose glycans, whereas the insoluble fraction containing the nuclear membrane was enriched with glycoproteins containing LacNAcs (galactose and N-acetylglucosamine disaccharide). Lectins that specifically bind to these highly abundant nuclear membrane glycans were then identified by searching the National Cancer Institute's Center for Cancer Research Database of Anti-glycan Reagents (DAGR) for lectins that bind to the LacNAc epitope. These lectins include Amaranthus caudatus lectin, Datura stramonium lectin, Erythrina crista-galli lectin (ECL), Lycopersicon esculentum lectin, Maackia amurensis agglutinin I, and Ricinus communis agglutinin I (RCA), e.g., RCA120, which are shown in Table 3. Erythrina crista-galli lectin (ECL) and Ricinus communis agglutinin (RCA) preferentially bind to galactose, N-acetylgalactosamine (GalNAc), and N-Acetyllactosamine (LacNAc) and were selected for use for handling and isolating cell nuclei to demonstrate the methods described herein.

### Example 4: Production of Lectin-Conjugated Beads

Magnetic beads were conjugated to lectins for use in isolation of cell nuclei as described herein.

TABLE 4
Formulations of buffers for preparing lectin-conjugated beads

| Buffer | Components |
|---|---|
| Bead wash buffer | 1x PBS |
| Bead conjugation buffer | 1X PBS containing 0.01% Tween-20 |
| Bead activation buffer | 20 mM HEPES, 10 mM KCl, |
| | 10 mM CaCl₂ 1 mM MnCl₂ |
| Nuclear extraction buffer | 20 mM HEPES, 10 mM KCl, |
| | 20% Glycerol, 1 mM MnCl₂, |
| | 0.1% Triton X-100 |
| EDTA-free Isotonic buffer | 10 mM Tris pH 8.0, 15 mM NaCl, |
| | 60 mM KCl |

Formulations of buffers used for the preparation of lectin-conjugated beads can be found in Table 4.

To prepare lectin-conjugated beads, 250 μl of magnetic streptavidin beads at a concentration of 4 mg/mL (1000 μg of magnetic beads) were pipetted into a 1.5 mL tube. The tube was then placed on a magnetic rack for 1 minute until the magnetic beads were collected to the side of the tube. The supernatant was carefully removed without disturbing the magnetic beads. To wash the beads, the tube was removed from the magnetic rack and the beads were resuspended in 500 μl of Bead wash buffer. The tube was then placed on a magnetic rack for 1 minute until the magnetic beads were collected to the side of the tube. The wash step was repeated two more times. After the three washes, the magnetic beads were resuspended into 250 μl (the original bead volume) of Bead conjugation buffer. 25 μl of biotinylated lectin suspension (such as biotinylated ECL or biotinylated RCA) at a concentration of 5 mg/mL was added to the tube. The tube was gently shaken (300-350 RPM) for 30 minutes at room temperature and placed on a magnetic rack for 1 minute until the magnetic beads were collected to the side of the tube. The beads were then washed as described previously and resuspended into 250 μl of Bead conjugation buffer and used for downstream applications or stored at 4° C. for future use.

### Example 5: Isolation of Cell Nuclei and Nuclear Fractions Using Lectin-Coated Magnetic Beads

Cell nuclei and nuclear fractions were prepared using lectin-coated magnetic beads.

TABLE 5
Formulations of buffers used for isolating cell nuclei

| Buffer | Components |
|---|---|
| Bead Activation Buffer | 20 mM HEPES, 10 mM KCl, |
| | 10 mM CaCl₂, 1 mM MnCl₂ |
| Nuclear Extraction Buffer | 20 mM HEPES, 10 mM KCl, |
| | 20% Glycerol, 1 mM MnCl₂, |
| | 0.1% Triton X-100 |
| EDTA-free Isotonic Buffer | 10 mM Tris pH 8.0, 15 mM NaCl, |
| | 60 mM KCl |
| Euchromatin Buffer (low | 10 mM Tris pH 8.0, 250 mM NaCl, |
| salt buffer) | 1 mM EDTA pH 8.0 |
| Heterochromatin Buffer | 10 mM Tris pH 8.0, 600 mM NaCl, |
| (high salt buffer) | 1 mM EDTA pH 8.0 |

Formulations of buffers used for the isolation of these cell components can be found in Table 5.

Lectin-conjugated magnetic beads were prepared as described in Example 2, and the beads were activated prior to their use. To activate the beads, 20 μL of beads per million cells was transferred to a 1.5 mL polypropylene tube. For example, 1200 μL of beads was transferred for a total of 12 samples that each contained 5 million cells. The tube containing the beads was placed on a magnetic tube rack and allowed to sit for 1 minute until the beads were collected to the side of the tube by the magnet. The supernatant was then removed and discarded. The tube was removed from the magnet and washed with Bead Activation Buffer three times. For each wash, the beads were fully resuspended into 1200 μL of Bead Activation Buffer. Then, the tube containing the beads was placed on a magnetic tube rack and allowed to sit for 1 minute until the beads were collected to the side of the tube by the magnet. The supernatant containing the Bead Activation Buffer was removed and discarded. After the three washes, the lectin-conjugated beads were resuspended into 1200 μL of Bead Activation Buffer and ready for use.

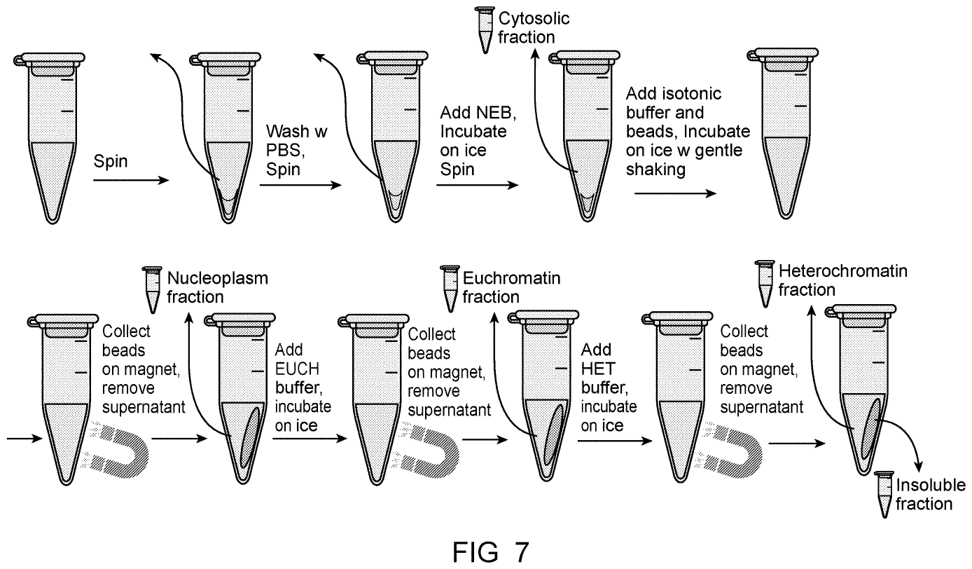

As illustrated in FIG. 7, to isolate cell nuclei and nuclear fractions using the activated lectin-coated magnetic beads, cells were collected into 1.5 mL polypropylene tubes and centrifuged at 400 RCF for 5 minutes at 4° C. The supernatant was removed and discarded. The cell pellets were resuspended in 1 mL of ice cold 1×PBS then centrifuged at 400 RCF for 5 minutes at 4° C. The supernatant was removed and discarded. Each pellet was resuspended in 200 μL of Nuclear Extraction Buffer (NEB) and incubated for 10 minutes at 4° C. After the incubation, the samples containing the isolated nuclei were centrifuged at 400 RCF for 5 minutes at 4° C. The supernatant was removed and retained as the Cytosolic Fraction.

The pellet containing the nuclei was resuspended in 200 μL of EDTA-free Isotonic Buffer. Activated lectin-conjugated magnetic beads were added to each sample at a volume of 20 μL per million cells. In this example, 100 μL of activated beads was added to each sample, which contained 5 million cells. The activated beads were mixed thoroughly with the pellet and the Isotonic Buffer by pipetting up and down. The samples were then incubated at 4° C. with gentle shaking for 20 minutes to allow for the cell nuclei to bind to the lectin-conjugated magnetic beads. The tubes containing the nuclei and beads suspension were placed onto a magnetic rack and allowed to sit for at least one minute for the beads to pellet and collect onto the side of the tube. The supernatant was carefully removed without disturbing the pellet and retained as the Nucleoplasm Fraction.

The tubes were then removed from the magnetic rack, and the pellets were resuspended in 200 μL of euchromatin buffer and incubated at 4° C. for 15 minutes. The tubes were then returned to the magnetic rack and allowed to sit for 1 minute for the beads to pellet and collect onto the side of the tube. The supernatant was carefully removed and retained as the Euchromatin Fraction.

The tubes were then removed from the magnetic rack, and the pellets were resuspended in 200 μL of heterochromatin buffer and incubated at 4° C. for 15 minutes. The tubes were then returned to the magnetic rack and allowed to sit for 1 minute for the beads to pellet and collect onto the side of the tube. The supernatant was carefully removed and retained as the Heterochromatin Fraction. The remaining pellet containing the insoluble fraction was stored for future use. The insoluble fraction contains cell debris, including chromatin and insoluble chromatin-associated proteins, and can be further analyzed, e.g., for histone post-translational modifications, or for isolating nucleic acids for further analysis.

### Example 6: Use of Erythrina crista-Galli Lectin (ECL) and Ricinus communis Agglutinin (RCA) for Nuclear Protein Extraction

To compare the use of ECL or RCA-conjugated magnetic beads with ConA-conjugated magnetic beads for cell nuclei isolation, the nucleoplasm, euchromatin, and heterochromatin fractions were extracted from 4 million MM1. S cells, as described in Example 5 above.

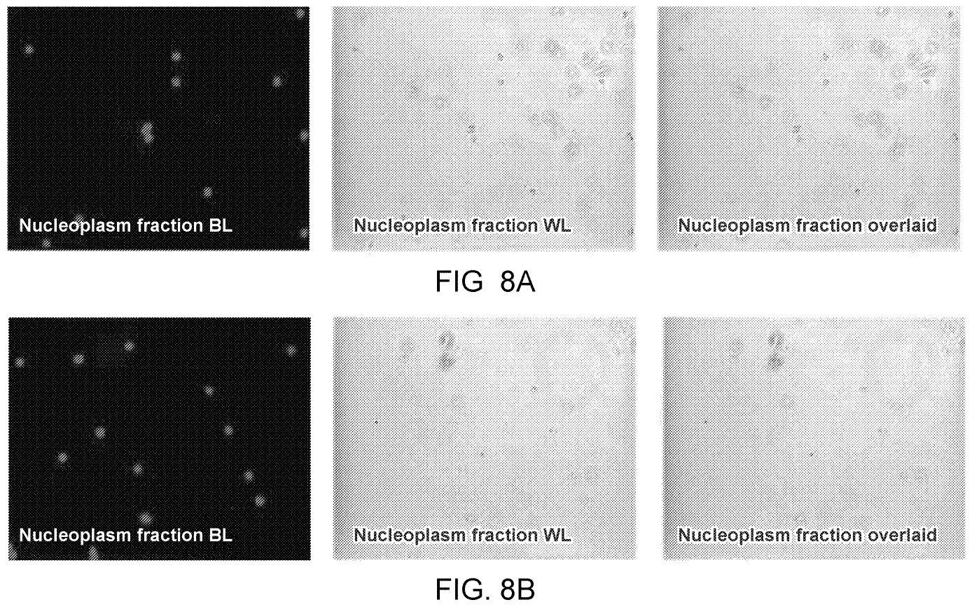

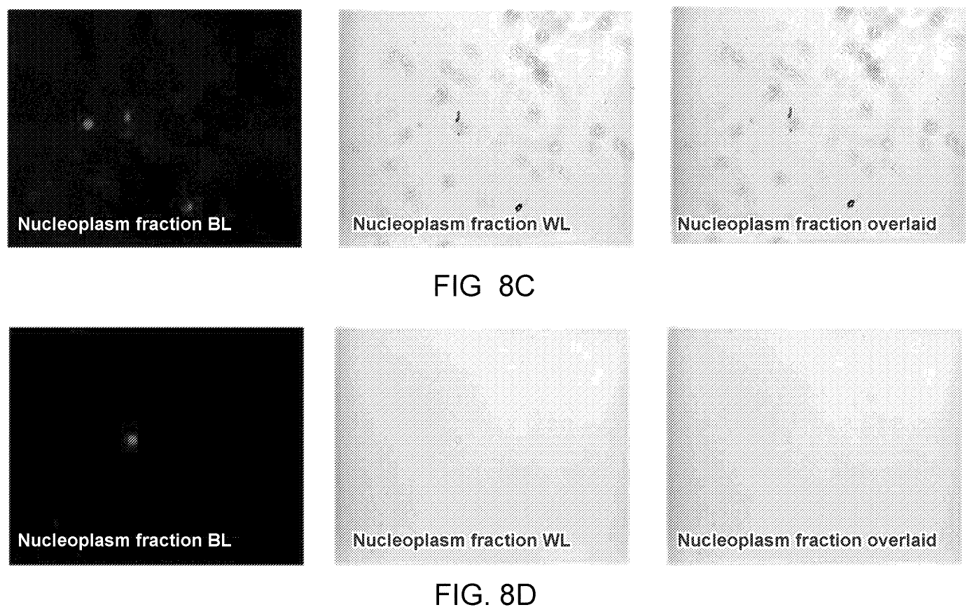

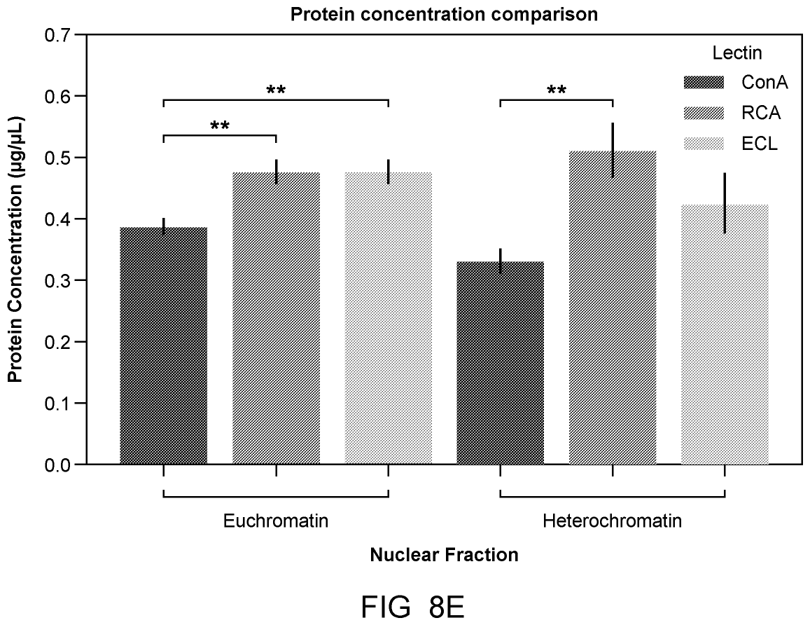

Images of the nucleoplasm fractions were taken using an EVOS FLoid imaging system. As shown in the blue light images in FIGS. 8A-8D, the nucleoplasm fractions extracted using ConA-conjugated magnetic beads (FIGS. 8A-8B) have more left-behind nuclei when compared to that of RCA-conjugated magnetic beads (FIG. 8C) and ECL-conjugated magnetic beads (FIG. 8D). Additionally, the brightfield images in FIGS. 8A-8D show fewer nuclei left behind with the RCA and ECL-conjugated magnetic beads, which demonstrates these lectins are more effective at binding to and moving nuclei than ConA.

The amount of protein isolated in each of the euchromatin and heterochromatin fractions using the ConA, RCA, and ECL-conjugated magnetic beads was determined using a 660-nm Protein Assay. As shown in FIG. 8E, both the euchromatin fractions extracted by RCA and ECL and the heterochromatin fractions extracted using RCA-conjugated magnetic beads yielded significantly higher amounts of protein compared to that of ConA. These results show that ECL and RCA are more efficient than ConA at nuclear protein extraction. (p-values of <0.01 denoted by “**”).

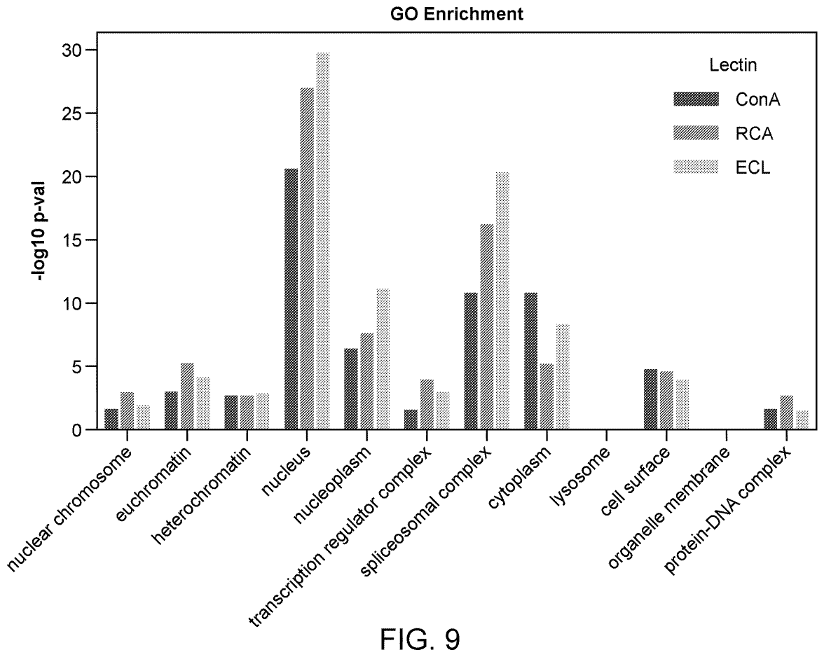

The combined chromatin (combining equal volumes of euchromatin and heterochromatin fraction) isolated using ConA, ECL, or RCA-conjugated magnetic beads were analyzed by LC-MS, and GO (gene ontology) enrichment analysis was used to evaluate the protein content of each sample. As shown in FIG. 9, RCA- and ECL-conjugated beads successfully enriched nuclear proteins, with enrichment of proteins associated with the nuclear chromosome, euchromatin, heterochromatin, nucleus, nucleoplasm, and protein-DNA complex. Additionally, RCA and ECL-conjugated magnetic beads led to higher enrichment of proteins associated with the nucleus compared to that of ConA-conjugated magnetic beads. Importantly, use of ConA resulted in higher enrichment for cytoplasmic and cell-surface proteins compared to ECL and RCA, demonstrating that ECL and RCA are superior lectins for use in isolating cell nuclei for protein extraction.

### Example 7: Automated Nuclear Protein Extraction Using Lectin-Coated Magnetic Beads in a 96-Well Format

To test automation and applicability to drug discovery for high-throughput nuclear protein extraction and fractionation using ECL-conjugated magnetic beads, a Thermo Scientific KingFisher™ Apex System was used to extract the nucleoplasm, chromatin, and insoluble fractions from treated THP-1 cells.

In the present example, THP-1 cells were collected and seeded in a V-bottom 96-well plate with wells containing the drugs dBET6, VTP56345345, or DMSO. Cells in 170 μL of media (RPMI-1 containing 10% fetal bovine serum and 1×penicillin/streptomycin) were transferred into each well of the plate. The cells were incubated for 4 hours at 37° C. After the treatment incubation, the cells were transferred to a 200 μL KingFisher™ plate and then centrifuged at 400 RCF for 5 minutes at 4° C. The supernatant was removed and discarded. 170 μL of ice-cold 1×PBS was added to each well of the treated cells and then centrifuged at 400 RCF for 5 minutes at 4° C. The supernatant was removed and discarded. 140 μL of NEB was added to each well and the cells were pipetted up and down at least three times to mix. 10 μL of ECL-conjugated and activated (as described in Example 6 above) beads were added to each well. The cell and bead suspension was pipetted up and down to mix. The plate was covered with a clear plastic seal and incubated with gentle shaking (450 RPM) on a Thermomixer for 20 minutes at 4° C.

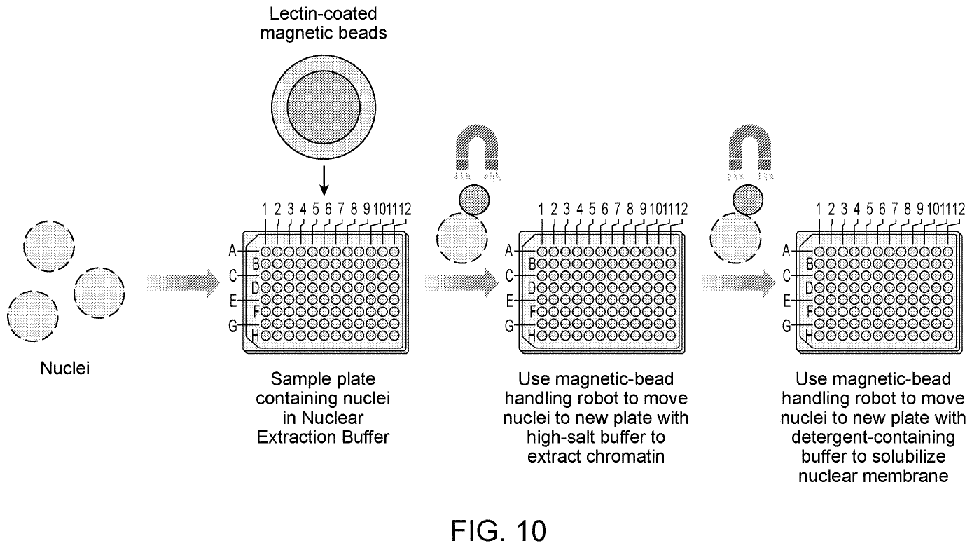

After incubation, the automated chromatin extraction by salt separation (ChESS) program was then initiated, which directed the KingFisher™ system to rotate through the loaded plates in sequential order, as shown in FIG. 10. The 96-well plate containing the samples was loaded into the KingFisher™ system, along with three other plates. The first 96 deep-well plate contained 125 μL per well of isotonic buffer (10 mM Tris pH 8.0, 15 mM NaCl, 60 mM KCl) to extract nucleoplasm and the second contained 125 μL per well of heterochromatin extraction buffer (10 mM Tris pH 8.0, 600 mM NaCl, 1 mM EDTA pH 8.0) to extract chromatin. Lastly, the third plate, a KingFisher™ 200 μL plate, contained 125 μL per well of insoluble buffer (1% sodium dodecyl sulfate, 10 mM MgCl2, in phosphate-buffered saline). KingFisher™ 96 tip combs (Thermo Fisher Scientific; Cat #97002534) for deep-well magnets were assembled with the 96 deep-well plates as directed by manufacturer instructions and used for sample transfer. The three plates containing isolated nuclear fractions (nucleoplasm, chromatin, or insoluble) were retained for downstream processing at the end of the KingFisher™ program run.

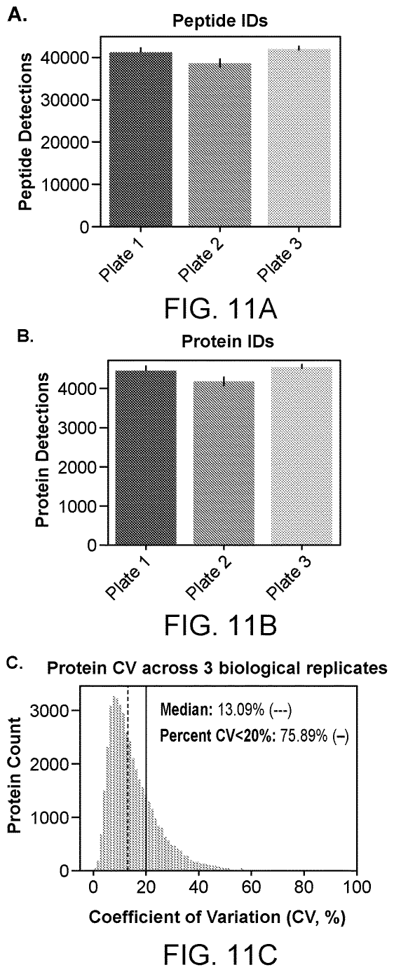

The above process was repeated two additional times for a total of three biological replicates. The chromatin fractions of these plates were digested with trypsin and analyzed by LC-MS using a Bruker timsTOF Ultra. As shown in FIGS. 11A-11B, high peptide and protein detections were observed across conditions and across the plate replicates, and the median coefficient of variation (CV) was calculated to be less than 13.09% for all proteins across plates, and the total percentage of proteins with a CV of less than 20% was 75.89% (FIG. 11C). Together, these results demonstrate the protocol's robustness in varying sample environments and between replicates.

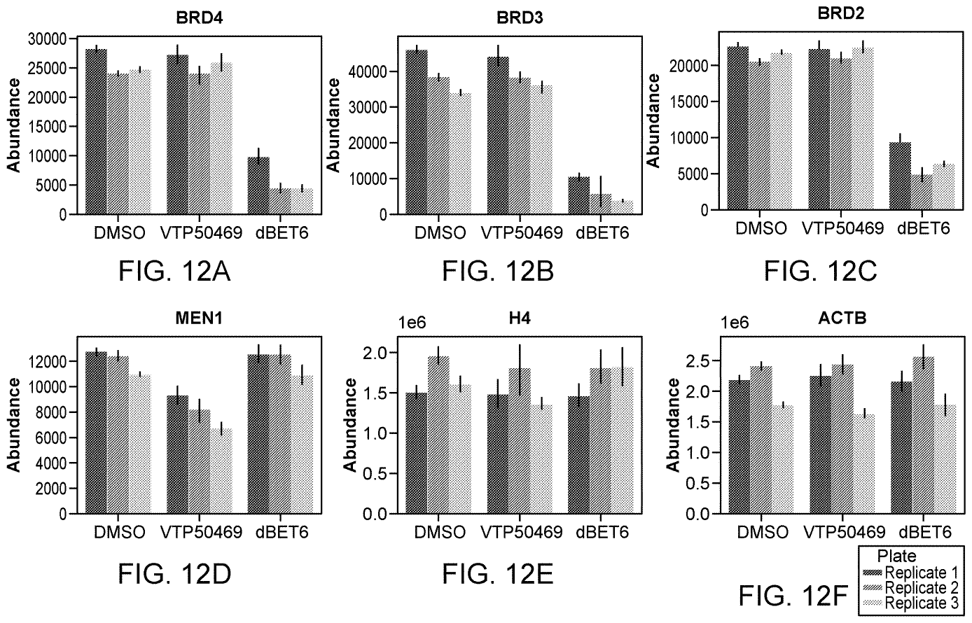

In addition, FIGS. 12A-12C show that samples treated with the compound dBET6 have a decreased expression of the BRD family proteins, which are the validated targets of that compound. FIG. 12D shows that samples treated with the compound VTP50469 have a decreased level of MEN1 expression, which is the validated target protein of that compound. Importantly, control proteins such as H4 and ACTB demonstrate stable expression, remaining largely unchanged between treatment conditions (FIGS. 12E-12F). These results in FIGS. 12A-12F illustrate that this protocol is capable of characterizing the accurate biological impact of a compound on the protein content of the nucleus, and these effects are captured across replicates with low technical variation. Thus, these figures demonstrate that automated nuclear extraction of proteins using lectin coated magnetic beads can be performed reproducibly in a 96-well format, and be applied to high-throughput proteomics for drug discovery applications or other applications requiring large-scale processing of the nuclear and chromatin-associated proteome.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

### LIST OF REFERENCES

Varnavides et. al. In Search of a Universal Method: A Comparative Survey of Bottom-Up Proteomics Sample Preparation Methods. J Proteome Res. 2022 Oct. 7; 21 (10): 2397-2411

We/I claim:

1. A method of extracting proteins from nuclei and chromatin, comprising contacting nuclei and/or chromatin with a solution comprising a volatile salt, separating insoluble chromatin from the solution, and collecting the solution supernatant, wherein the solution supernatant comprises proteins from nuclei and/or chromatin.

2. The method of claim 1, further comprising analyzing the extracted proteins by mass spectrometry.

3. The method of claim 2, wherein the volatile salt is not removed from the extracted proteins before the analysis by mass spectrometry.

4. The method of any one of claims 1-3, wherein the proteins comprise peptide fragments of full-length proteins.

5. The method of any one of claims 1-4, further comprising separating nuclei from cells before contacting nuclei and/or chromatin with the volatile salt.

6. The method of any one of claims 1-5, comprising isolating samples enriched in nucleoplasm associated proteins, euchromatin associated proteins, and/or heterochromatin associated proteins from cells, the method comprising:
(a) lysing the cells;
(b) separating nuclei from the cells following (a), optionally obtaining cytoplasmic proteins; and
(c) extracting nucleoplasm associated proteins, euchromatin associated proteins, and/or heterochromatin associated proteins from the separated nuclei by contacting nuclei and/or chromatin with a solution comprising a volatile salt, separating insoluble chromatin from the solution, and collecting the solution supernatant, wherein the solution supernatant comprises nucleoplasm associated proteins, euchromatin associated proteins, and/or heterochromatin associated proteins.

7. The method of any one of claims 1-5, comprising isolating samples enriched in nucleoplasm associated proteins, euchromatin associated proteins, and/or heterochromatin associated proteins from cells, the method comprising:
(a) contacting cells with a detergent solution to lyse the cells;
(b) separating nuclei from the cells following (a), optionally obtaining cytoplasmic proteins; and
(c) sequentially extracting the nucleoplasm associated proteins, euchromatin associated proteins, and/or heterochromatin associated proteins from the separated nuclei as follows:
(i) re-suspending the nuclei of (b) in a first solution comprising a first concentration of a volatile salt to extract nucleoplasm associated proteins;
(ii) separating insoluble chromatin after (i);
(iii) collecting a first supernatant liquid after (ii), wherein the first supernatant liquid is enriched for nucleoplasm associated proteins;
(iv) re-suspending the insoluble chromatin of (ii) in a second solution comprising a second concentration of the volatile salt to extract euchromatin associated proteins;
(v) separating insoluble chromatin after (iv);
(vi) collecting a second supernatant liquid after (v), wherein the second supernatant liquid is enriched for euchromatin associated proteins;
(vii) re-suspending the insoluble chromatin of (v) in a third solution comprising a third concentration of the volatile salt to extract heterochromatin associated proteins;
(viii) separating insoluble chromatin after (vii); and
(ix) collecting a third supernatant liquid after (viii), wherein the third supernatant liquid is enriched for heterochromatin associated proteins,
thereby isolating a first sample enriched in nucleoplasm associated proteins, a second sample enriched in euchromatin associated proteins, and a third sample enriched in heterochromatin associated proteins from the cells.

8. The method of any one of claims 1-7, wherein the cells, nuclei, and chromatin are not contacted with a non-volatile salt.

9. The method of any one of claims 1-8, wherein the volatile salt is not removed from the supernatant liquid.

10. The method of any one of claims 1-9, wherein the volatile salt is ammonium acetate, ammonium bicarbonate, trimethylammonium acetate, or trimethylammonium bicarbonate.

11. The method of claim 10, wherein the volatile salt is ammonium acetate.

12. The method of any one of claims 6-11, wherein:
(a) the first solution comprises 50-150 mM of the volatile salt;
(b) the second solution comprises 200-500 mM of the volatile salt; and
(c) the third solution comprises 500 mM-1000 mM of the volatile salt.

13. The method of any one of claims 1-11, wherein the solution(s) comprises 500-1000 mM of the volatile salt.

14. The method of claim 13, wherein the solution(s) comprises 600 mM of the volatile salt.

15. The method of claim 13 or claim 14, wherein the solution(s) further comprises 0.1-1% of a detergent.

16. The method of claim 15, wherein the detergent is selected from Triton X-100, NP-40, or SDS.

17. A method for comparing the proteins present in the nucleus and/or chromatin-associated proteins of two or more different cell populations, comprising:
(a) extracting proteins from the nucleus and/or chromatin of a first cell population according to the method of any one of claims 1-16;
(b) extracting proteins from the nucleus and/or chromatin of a second cell population according to the method of any one of claims 1-16; and
(c) analyzing the proteins obtained from the first cell population and the second cell population,
thereby comparing the proteins present in the nucleus and/or chromatin-associated proteins of the two or more different cell populations.

18. The method of claim 17, wherein the first cell population and the second cell population were exposed to different conditions.

19. The method of claim 18, wherein the different conditions comprise one or more of: different growth or culture conditions; different environmental conditions; and presence or absence of, or different, genomic or cellular modifications.

20. A method of extracting proteins from nuclei, comprising contacting nuclei bound to a solid support with a solution comprising a volatile salt, and then separating the nuclei bound to the solid support from the solution, wherein following the contacting, the solution comprises nuclear and/or chromatin-associated proteins.

21. The method of claim 20, wherein the volatile salt is ammonium acetate.

22. The method of claim 20 or claim 21, further comprising analyzing the extracted proteins by mass spectrometry.

23. The method of claim 22, wherein the volatile salt is not removed from the extracted proteins before the analysis by the mass spectrometry.

24. The method of any one of claims 20-23, wherein the proteins comprise peptide fragments of full-length proteins.

25. The method of any one of claims 20-24, further comprising separating nuclei from cells before contacting the nuclei with the volatile salt.

26. The method of any one of claims 20-25, comprising preparing samples enriched in nucleoplasm associated proteins, euchromatin associated proteins, and/or heterochromatin associated proteins from cells, the method comprising:
(a) contacting cells with a detergent solution to lyse the cells;
(b) binding the nuclei to a solid support and separating nuclei from the cells following (a), optionally obtaining cytoplasmic proteins; and
(c) sequentially extracting the nucleoplasm associated proteins, euchromatin associated proteins, and/or heterochromatin associated proteins from the separated nuclei as follows:
(i) contacting the nuclei bound to the solid support following (b) with a first solution comprising a first concentration of a volatile salt to extract nucleoplasm associated proteins;
(ii) separating the nuclei bound to the solid support from the first solution, wherein the first solution comprises nucleoplasm associated proteins following (i);
(iii) contacting the nucleic bound to the solid support with a second solution comprising a second concentration of the volatile salt to extract euchromatin associated proteins;
(iv) separating the nuclei bound to the solid support from the second solution, wherein the second solution comprises euchromatin associated proteins following (iii);
(v) contacting the nucleic bound to the solid support with a third solution comprising a third concentration of the volatile salt to extract heterochromatin associated proteins; and
(vi) separating the nuclei bound to the solid support from the third solution, wherein the second solution comprises heterochromatin associated proteins following (v),
thereby isolating a first sample enriched in nucleoplasm associated proteins, a second sample enriched in euchromatin associated proteins, and a third sample enriched in heterochromatin associated proteins from the cells.

27. The method of any one of claims 20-26, wherein the nuclei are not contacted with a non-volatile salt.

28. The method of any one of claims 20-27, wherein the volatile salt is not removed from the samples.

29. The method of claim 27 or claim 28, wherein the volatile salt is ammonium acetate, ammonium bicarbonate, trimethylammonium acetate, or trimethylammonium bicarbonate.

30. The method of claim 29, wherein the volatile salt is ammonium acetate.

31. The method of any one of claims 26-30, wherein:
(a) the first solution comprises 50-150 mM of the volatile salt;
(b) the second solution comprises 200-500 mM of the volatile salt; and
(c) the third solution comprises 500-1000 mM of the volatile salt.

32. A method for comparing the proteins present in the nucleus and/or chromatin-associated proteins of two or more different cell populations, comprising:
(a) extracting proteins from the nucleus and/or chromatin of a first cell population according to the method of any one of claims 20-31;
(b) extracting proteins from the nucleus and/or chromatin of a second cell population according to the method of any one of claims 20-31; and
(c) analyzing the proteins obtained from the first cell population and the second cell population,
thereby comparing the proteins present in the nucleus and/or chromatin-associated proteins of the two or more different cell populations.

33. The method of claim 32, wherein the first cell population and the second cell population were exposed to different conditions.

34. The method of claim 33, wherein the different conditions comprise one or more of: different growth or culture conditions; different environmental conditions; and presence or absence of, or different, genomic or cellular modifications.

35. The method of any one of claims 20-34, wherein the solid support comprises one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc) on the nuclei.

36. The method of claim 35, wherein the one or more lectin comprises Erythrina crista-galli lectin (ECL), Ricinus communis agglutinin (RCA), Lycopersicon esculentum lectin (LEL), Amaranthus caudatus lectin, Maackia amurensis agglutinin I, Datura Stramonium lectin (DSL), Solanum tuberosum lectin, soybean agglutinin, Vicia villosa lectin, and/or Wisteria floribunda lectin.

37. The method of claim 35 or claim 36, wherein the solid support is a bead.

38. The method of claim 37, wherein the bead is a magnetic bead.

39. The method of any one of claims 20-38, wherein the nuclei are contacted with the solid support while in a sample comprising cells, cell lysate, and/or other non-nuclear cellular components.

40. The method of claim 38, wherein the solid support and associated nuclei are transferred or removed using a magnet.

41. The method of any one of claims 20-40, wherein the method is a high-throughout method, wherein the nuclei are present in the first solution within multiple wells of a sample plate, the solid support is a magnetic bead, and nuclei bound to the solid support are transferred or removed from the multiple wells using magnets.

42. The method of claim 41, wherein following transfer or removal from the multiple wells comprising the first solution, the nuclei are placed into a second solution within multiple wells of a sample plate, and the nuclei bound to the solid support are then transferred or removed from the multiple wells using magnets.

43. The method of claim 42, wherein following transfer or removal from the multiple wells comprising the second solution, the nuclei are placed into a third solution within multiple wells of a sample plate, and the nuclei bound to the solid support are then transferred or removed from the multiple wells using magnets.

44. The method of claim 43, wherein the first solution is an isotonic solution to extract nucleoplasm proteins, the second solution is a low salt solution to extract euchromatic proteins, and the third solution is a high salt solution to extract heterochromatin proteins.
